(19) United States
(12) Patent Application Publication
(10) Pub. No.: US 2012/0039919 A1
(43) Pub. Date: Feb. 16, 2012

(54) DIABETES DIAGNOSTIC, PROPHYLACTIC, AND THERAPEUTIC COMPOSITIONS AND METHODS

(75) Inventors: Lijun Yang, Gainesville, FL (US); Vijay Koya, Hershey, PA (US); Shi-Wu Li, Gainesville, FL (US)

(73) Assignee: UNIVERSITY OF FLORIDA RESEARCH FOUNDATION, INC., Gainesville, FL (US)

(21) Appl. No.: 13/204,357

(22) Filed: Aug. 5, 2011

Related U.S. Application Data
(63) Continuation-in-part of application No. PCT/US2010/000344, filed on Feb. 5, 2010.
(60) Provisional application No. 61/150,295, filed on Feb. 5, 2009.

(51) Int. Cl. A61K 39/00; C07H 21/00; C12N 15/63; C12N 5/10; A61K 31/7088; A61P 37/02; C12N 1/00; C12N 1/21; C12N 1/19; A61P 3/10; A61P 37/04; A61P 37/06; C07K 14/00; G01N 33/53

(52) U.S. Cl. 424/184.1; 530/350; 530/324; 536/23.1; 435/320.1; 435/325; 435/7.92; 514/44 R; 436/501; 435/243; 435/252.33; 435/254.21; 435/348; 435/357; 435/367; 435/365

(57) ABSTRACT

As described below, the present invention features compositions and methods featuring Pdx-1 polypeptides and fragments thereof for the diagnosis, prevention and treatment of diabetes.

### CROSS-REFERENCE TO RELATED APPLICATION

This application is a continuation-in-part of International Application No. PCT/US2010/000344, filed on Feb. 5, 2010 and published as WO 2010/090758 on Aug. 12, 2010; which claims the benefit of U.S. Provisional Application No. 61/150,295, filed on Feb. 5, 2009, the contents of which are incorporated herein in their entireties by reference.

### STATEMENT OF RIGHTS TO INVENTIONS MADE UNDER FEDERALLY SPONSORED RESEARCH

The work was supported by the following grants from the National Institutes of Health, Grant Nos: NIDDK DK071831 and DK64054. The Government has certain rights in the invention.

### BACKGROUND OF THE INVENTION

Diabetes is a disorder characterized by elevated blood glucose (hyperglycemia). Type 1 diabetes is characterized by autoimmune destruction of insulin-producing beta-cells in the pancreas. In type II diabetes, the beta cells of the pancreas produce insulin, but the body is resistant to the insulin produced. Sixteen million Americans have diabetes, although many of these diabetics are undiagnosed. Early identification of those having a propensity to develop diabetes is critical because of the potential long term complications of the disease, which can have devastating effects on the kidneys, eyes, heart, blood vessels and nerves. Currently, no reliable method exists to predict who will develop diabetes.

### SUMMARY OF THE INVENTION

As described below, the present invention features compositions and methods for the diagnosis, prevention and treatment of diabetes.

In one aspect, the invention generally provides an isolated Pdx-1 peptide containing an amino acid sequence having about 70, 75, 80, 85, 90, 95, or 100% identity to a Pdx-1 C terminus. In one embodiment, the Pdx-1 C terminus contains about 50-200 (e.g., 50, 75, 100, 125, 150, 200) amino acids from the C terminus of Pdx-1. In another embodiment, the Pdx-1 C terminus contains or consists essentially of amino acids 150-200 or 200-283/284 of Pdx-1.

In another embodiment, the peptide lacks DNA binding activity. In another embodiment, the peptide has a reporter moiety.

In another aspect, the invention provides an isolated Pdx-1 peptide (e.g., human Pdx-1) containing an amino acid sequence identified as binding an autoantibody.

In yet another aspect, the invention provides an isolated Pdx-1 peptide consisting essentially of amino acids 200-283/284 of Pdx-1 or amino acids 150-200 of Pdx-1. In a related aspect, the invention provides an isolated nucleic acid molecule encoding a Pdx-1 peptide of any previous aspect. In one embodiment, the nucleic acid molecule encodes a Pdx-1 C terminus containing at least about 50-200 amino acids from the C terminus of Pdx-1. In another embodiment, the Pdx-1 C terminus contains amino acids 150-200 or 200-283/284 of Pdx-1. In yet another embodiment, the isolated Pdx-1 nucleic acid molecule is a nucleotide sequence encoding amino acids 150-200 or 200-283/284 of Pdx-1.

In another related aspect, the invention provides an expression vector that contains a nucleic acid molecule encoding a Pdx-1 peptide delineated herein positioned for expression.

In one embodiment, the vector further contains a promoter suitable for expression in a cell (e.g., a bacterial or mammalian host cell). In various embodiments, the promoter is a cytomegalovirus (CMV) promoter. In another embodiment, the expression vector encodes a Pdx-1 peptide fused to a reporter construct. In various embodiments, the reporter is luciferase.

In yet another related aspect, the host cell (e.g., a prokaryotic or eukaryotic cell, such as an E. coli cell, or a mammalian rodent or human cell) contains an expression vector delineated herein.

In still another aspect, the invention provides a pharmaceutical composition containing an effective amount of a Pdx-1 peptide or nucleic acid molecule delineated herein and a pharmaceutically acceptable excipient.

In a related aspect, the invention provides an immunogenic composition or vaccine containing a Pdx-1 polypeptide or fragment thereof in an amount sufficient to modulate an immune response and a pharmaceutically acceptable excipient. In one embodiment, the immunogenic composition or vaccine contains about 1-1000 μg of a Pdx-1 polypeptide or a fragment thereof. In another embodiment, the immunogenic composition or vaccine contains about 50, 100, 200, or 500 μg of Pdx-1 polypeptide or a fragment thereof.

In another related aspect, the invention provides a pharmaceutical composition containing an effective amount of a Pdx-1 peptide having immunomodulatory activity in a pharmaceutically acceptable carrier. In one embodiment, the Pdx-1 peptide has autoantibody binding activity. In another embodiment, the Pdx-1 peptide induces immunotolerance in a subject. In still another embodiment, the composition further contains an adjuvant.

In another aspect, the invention provides a method for identifying a subject as having or having a propensity to develop diabetes, the method involves detecting a Pdx-1 autoantibody in a biological sample (e.g., a tissue sample or biological fluid sample) of the subject. In one embodiment, the detecting is done on two or more occasions and an increase in Pdx-1 specific antibodies is a diagnostic indicator of pre-diabetes or diabetes. In another embodiment, the detecting is by radioimmunoassay, ELISA, reporter assay, or luciferase reporter assay. In yet another embodiment, the subject is related to a subject diagnosed as having type 1 diabetes.

In another aspect, the invention provides a method for monitoring pre-diabetes or diabetes in a subject, the method involves detecting a Pdx-1 specific antibody in a biological sample of the subject. In one embodiment, a decrease in Pdx-1 specific antibody level relative to a reference indicates an improvement in pre-diabetes or diabetes in the subject. In another embodiment, the biological sample is a tissue sample or biological fluid sample (e.g., blood, serum, plasma or urine). In another embodiment, the method detects the presence of absence of the antibody. In another embodiment, an increase in Pdx-1 antibody level is indicative of an increase in beta cell destruction.

In another aspect, the invention provides a method of preventing or treating pre-diabetes or diabetes in a subject, the method involves administering to the subject an effective amount of a Pdx-1 peptide of any of claims 1-7 or a polynucleotide encoding the peptide.

In yet another aspect, the invention provides a method of preventing or treating pre-diabetes or diabetes in a subject, the method involves administering to the subject an effective amount of a Pdx-1 polypeptide for a time and in an amount sufficient to modulate an immune response in a subject identified as having Pdx-1 autoantibodies.

In still another aspect, the invention provides method of suppressing an autoimmune response associated with diabetes in a subject, the method involves administering to a subject identified as having an increase in Pdx-1, GAD65, IA-2, and/or insulin autoantibodies an effective amount of a Pdx-1 polypeptide or peptide.

In a related aspect, the invention provides a method of inducing immunological tolerance in a subject, the method involves administering to a subject identified as having an increase in Pdx-1, GAD65, IA-2, and/or insulin autoantibodies an effective amount of a Pdx-1 polypeptide or peptide.

In another related aspect, the invention provides method of treating or preventing a Pdx-1 specific immune response in a subject, the method involves identifying the subject as having or having a propensity to develop Pdx-1 autoantibodies, and administering to the subject a Pdx-1 polypeptide or peptide having immunomodulatory activity.

In another aspect, the invention provides a method of treating or preventing pre-diabetes or diabetes, the method involves administering to a subject in need of such treatment an effective amount of a pharmaceutical composition containing an expression vector containing a nucleic acid molecule encoding a Pdx-1 peptide. In one embodiment, the subject is identified as having an increase in Pdx-1 autoantibodies relative to a normal control subject or relative to the level present in the subject at an earlier time point

In another aspect, the invention provides a method of inducing immunological tolerance in a subject, the method involves identifying the subject as having GAD65, IA-2, and/or insulin autoantibodies; and administering to the subject an effective amount of a Pdx-1 polypeptide or a nucleic acid molecule encoding a Pdx-1 polypeptide or peptide.

In another aspect, the invention provides an array containing an isolated Pdx-1 polypeptide or fragment thereof capable of binding a Pdx-1 autoantibody. In one embodiment, the Pdx-1 fragment is a peptide of a previous aspect or otherwise delineated herein. In one embodiment, the array further contains GAD65, IA-2, and/or insulin polypeptides or fragments thereof.

In another aspect, the invention provides a kit for use in identifying a subject as having a propensity to develop diabetes, the kit containing a peptide of any previous aspect or delineated herein, and written instructions for the use of the kit in diagnosing diabetes. In another embodiment, the kit further contains a means for identifying a GAD65, IA-2, and/or insulin autoantibody, such as a GAD65, IA-2, and/or insulin polypeptide or fragment thereof.

In various embodiments of any of the above aspects, the Pdx-1 C terminus contains about 50-200 (e.g., 50, 75, 100, 125, 150, 200) amino acids from the C terminus of Pdx-1. In another embodiment, the Pdx-1 C terminus contains or consists essentially of amino acids 150-200 or 200-283/284 of Pdx-1. In other embodiments of the invention, the peptide lacks DNA binding activity. In other embodiments of the invention, a decrease in Pdx-1 specific antibody level relative to a reference (e.g., a baseline level or prior measurement) indicates an improvement in pre-diabetes or diabetes in a subject. In another embodiment, the biological sample is a tissue sample or biological fluid sample (e.g., blood, serum, plasma or urine). In another embodiment, the method detects the presence of absence of a Pdx-1 autoantibody. In another embodiment, an increase in Pdx-1 autoantibody level is indicative of an increase in beta cell destruction. In various embodiments, autoantibodies are detected by an immunoassay, such as an ELISA, immunoprecipitation, Western blot, or radioimmunoassay. In further embodiment, a Pdx-1 autoantibody detection method further involves the step of detecting blood glucose level in the subject, conducting a fasting plasma glucose (FPG) test, oral glucose tolerance test, or random plasma glucose test, or detecting the presence or absence of GAD65, IA-2, and/or insulin autoantibodies in a biological fluid of the subject. Detection of an increase in Pdx-1 and/or GAD65, IA-2, and/or insulin autoantibodies is indicative of diabetes. In still other embodiments of the invention, the Pdx-1 polypeptide lacks a homeodomain, lacks DNA binding activity, and/or lacks transcriptional regulatory activity. In still other embodiments, the Pdx-1 polypeptide contains an epitope that binds a Pdx-1 autoantibody. In various embodiments of the above aspects or other aspects of the invention delineated herein, the Pdx-1 peptide or fragment contains or consists essentially of Pdx-1 amino acids 150-200 or 200-283/284. In still other embodiments, the Pdx-1 peptide contains about 50-200 amino acids from the C terminus of Pdx-1, where the range includes as its lower limit any integer between 50 and 199, and as its upper limit any integer between 51 and 200. Moreover, the range is intended to describe each integer falling within the range.

The invention provides compositions and methods for the diagnosis, prevention and treatment of diabetes. Other features and advantages of the invention will be apparent from the detailed description, and from the claims.

### DEFINITIONS

By “Pancreatic and Duodenal Homeobox—1 (Pdx-1) polypeptide” is meant a protein or fragment thereof having at least 70% homology to the sequence provided at GenBank Accession No. NP_{—}032840, AAI11593, or a sequence encoded by NM008814, and having immunomodulatory activity. In one embodiment, the Pdx-1 polypeptide has DNA binding activity or transcriptional regulation activity. In another embodiment, the Pdx-1 polypeptide lacks DNA binding or transcriptional regulatory activity. An exemplary murine Pdx-1 amino acid sequence is provided below:

| | |
|---|---|
| 1 | mnseeqyyaa tqlykdpcaf qrgpvpefsa nppaclymgr qpppppppqf tsslgsleqg |
| 61 | sppdispyev pplasddpag ahlhhhlpaq lglahpppgp fpngtepggl eepnrvqlpf |
| 121 | pwmkstkaha wkgqwaggay taepeenkrt rtaytraqll elekeflfnk yisrprrvel |
| 181 | avmlnlterh ikiwfqnrrm kwkkeedkkr ssgtpsgggg geepeqdcav tsgeellavp |
| 241 | plpppggavp pgvpaavreg llpsglsvsp qpssiaplrp qepr |

An exemplary human Pdx-1 amino acid sequence is provided below:

| | |
|---|---|
| 1 | mngeeqyyaa tqlykdpcaf qrgpapefsa sppaclymgr qpppppphpf pgalgaleqg |
| 61 | sppdispyev ppladdpava hlhhhlpaql alphppagpf pegaepgvle epnrvqlpfp |
| 121 | wmkstkahaw kgqwaggaya aepeenkrtr taytraqlle lekeflfnky isrprrvela |
| 181 | vmlnlterhi kiwfqnrrmk wkkeedkkrg ggtavggggv aepeqdcavt sgeellalpp |
| 241 | ppppggavpp aapvaaregr lppglsaspq pssvaprrpq epr |

Functional domains of human Pdx-1 are shown at FIG. 21. FIG. 22 provides an alignment of Pdx-1 polypeptides from various species.

By “Pdx-1 peptide” is meant a fragment of a Pdx-1 polypeptide having Pdx-1 immunomodulatory activity. In one embodiment, the peptide lacks a homeobox domain or lacks DNA binding activity.

By “Pdx-1 C terminus” is meant a fragment from the C terminal end of the full length Pdx-1 polypeptide.

In one embodiment, a Pdx-1 peptide comprises at least about 5, 10, 20, 30, 40, 50, 60, 70, 80, 90, 100, 110, 120 amino acids from the C-terminus of a Pdx-1 polypeptide. In another embodiment, the Pdx-1 peptide comprises amino acids 120-284, 150-284, 175-284, 200-284, 205-284, 210-284, 225-284, or 250-284 of murine Pdx-1. In another embodiment, the Pdx-1 peptide comprises amino acids 75-283, 100-283, 125-283, 125-275, 125-250, 120-200, 125-200, 135-200, 140-200, or 150-200. With regard to murine Pdx-1, the bottom of the range is any integer between 150 and 282 or between 200 and 282 and the top of the range is any integer between 150 and 283 or between 201 and 283. With regard to human Pdx-1, the bottom of the range is any integer between 100 and 282 or 150 and 199 and the top of the range is any integer between 102 and 283 or between 151 and 200.

By “Pancreatic and Duodenal Homeobox—1 (Pdx-1)” nucleic acid sequence is meant a nucleic acid sequence encoding PDX-1 or a peptide or fragment thereof. Exemplary pdx-1 nucleic acid sequences include BC111592 and NM_{—}008814. In one embodiment, the Pdx-1 nucleic acid sequence encodes amino acids 200-284 of Pdx-1 or amino acids 150-200 of Pdx-1. Exemplary Pdx-1 nucleic acid sequences include BC111592 and NM_{—}008814.

By “Pdx-1 biological activity” is meant transcriptional regulatory activity, DNA binding activity, specific binding of a Pdx-1 antibody, and/or immunomodulatory activity.

Such immunomodulatory activity refers to an increase or reduction in an immune response, such as a Pdx-1 specific immune response.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “autoantibody” is meant an antibody formed against an autoantigen. A subject having autoantibodies has an immune response targeting the subject's own tissues.

By “autoimmune response associated with diabetes” is meant an immune response against a beta islet cell antigen, pancreatic antigen, or other antigen associated with diabetes or an immune response associated with pancreatic cell death. Methods for measuring an autoimmune response associated with diabetes include any immunological or other method for assaying autoantibodies associated with diabetes (e.g., GAD65, IA-2 (also known as ICA512), and insulin autoantibodies).

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the object to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include bacterial invasion or colonization of a host cell.

By “DNA binding activity” is meant having a physicochemical affinity for DNA. DNA binding may be measured by any method known in the art, for example, electromobility shift assay, or a commercially available DNA binding assay, such as Clontech's Protein-DNA Binding Assay.

By “effective amount” is meant the amount of a required to ameliorate the symptoms of a disease relative to an untreated patient. The effective amount of active compound(s) used to practice the present invention for therapeutic treatment of a neurodegenerative disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in eukaryotic host organisms. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 71, 72, 73, 74, 75, 76, 77, 78, 79, 80, 81, 82, 83, 84, 85, 86, 87, 88, 90, or 100, 200, 300, 400, 500, 600, 700, 800, 900, or 1000 nucleotides or amino acids. Preferably, the fragment is from the C-terminus of Pdx-1.

By “homeodomain” is meant a highly conserved protein sequence motif comprising about 60 amino acids and containing a DNA-binding helix-turn-helix motif.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune response” is meant any response mediated by the immune system. An immune response includes, but is not limited to, may involve antibody production, induction of cell-mediated immunity, complement activation or development of immunological tolerance.

By “immunological tolerance” or “immunotolerance” is meant a reduction in an immune response to an antigen or failure to mount an immune response to an antigen.

By “immunomodulatory activity” is meant increasing or reducing an immune response. A reduction in an immune response following treatment with an autoantigen is indicative that immunotolerance has been induced.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “modulate” is meant an alteration, such as an increase or decrease.

By “polypeptide” is meant any chain of amino acids, regardless of length or post-translational modification (for example, glycosylation or phosphorylation).

By “pre-diabetes” is meant having a propensity to develop diabetes, or having any symptomatic or pathologic precursor to diabetes. Symptoms of diabetes are known in the art and include alterations in blood glucose levels, increased thirst, increased urination, or an abnormal result in a glucose challenge test. Subject having a propensity to develop diabetes have a genetic mutation associated with the disease (i.e., a mutation in Pdx-1), have a relative diagnosed as having diabetes, are obesity, or otherwise show physiological alterations associated with diabetes.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reference” is meant a standard or control condition.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1F are graphs showing Pdx-1 autoantibody levels in mice. FIG. 1A shows that Pdx-1 autoantibodies peak at about week ten in a mouse model of diabetes, the NOD mice. FIG. 1B shows that Pdx-1 autoantibody levels are increased in immune competent NOD mice relative to NOD SCID mice that lack competent immune systems, normal mice, and a mouse model of lupus. FIG. 1C shows the detection of Pdx 1 antibodies in NOD mice. Serum samples were collected from female NOD mice at various ages ranging from 8 to 23 weeks or NOD mice treated with daily injection of rPdx1 for 12 weeks. PAAs were assayed using ELISA at a serum dilution of 1:30. Positive value is defined 3 s.d. above the mean of the BALB/c control sera (Pre, pretreatment; Con, PBS-treated controls; Post-Rx, treated with rPdx1). FIG. 1D provides a comparison of PAAs in various strains of mice. Serum samples from various mouse strains at different ages were collected and PAAs were measured by ELISA and expressed as OD values at 450 nM using the 99th percentile of BALB/c control mice as the threshold definition of positivity. FIG. 1E provides titration curves of PAAs. Selected PAA-positive serum samples at the age of 10 weeks were serially diluted as indicated and OD values were determined by ELISA. Titer is defined as the last dilution giving positive OD reading above control. FIG. 1F is a graph showing levels of anti-Pdx1 antibody isotypes. Total anti-Pdx1 antibody activity (IgG-T) and anti-Pdx1 antibodies of the IgG1, IgG2b, and IgG3 isotypes in serum samples from NOD mice immunized with Pdx1 (immune sera, solid circles) and selected PAA-positive autoimmune sera (triangle) from NOD mice were assayed by ELISA (1:30 dilution). An OD of 0.10 was used as the positive cutoff value (X3s.d. above the mean of the BALB/c control sera).

FIGS. 2A and 2B show rPdx-1 separated by SDS-PAGE and stained with Coomassie blue and a Western blot of Pdx-1 probed with mouse anti-sera. FIG. 2A Western blotting. Purified recombinant rat Pdx1 protein (1 mg/lane) was separated on a 10% SDS-PAGE and stained with Coomassie blue or transferred to PVDF membrane. The membrane was probed with BALB/c or NOD mouse serum either positive (PAA+) or negative (PAA−) by ELISA, or with rabbit polyclonal anti-Pdx1 immune serum (rPdx1-IS) as a positive control. FIG. 2B Immunoprecipitation and western blotting. Rat insulinoma cells (INS-1) were labeled with [³⁵S]methionine overnight and cell lysate (0.5 mg) was incubated for 1 h at 4° C. with preformed immune complexes by incubating 10 ml mouse serum with 50 ml protein A/G-Sepharose overnight at 4° C. [³⁵S]-labeled proteins were separated by SDS-PAGE, fluorographed, and the dried gel was exposed to X-ray film at −80° C. for 7 days. In parallel, unlabeled INS-1 cell lysate was subjected to immunoprecipitation with the same mouse sera. The immune complexes were separated by SDS-PAGE, transferred to PVDF membrane, and probed with rabbit anti-Pdx1 polyclonal antibodies (1:2000). Lanes 1, BALB/c; 2, Pdx1-treated NOD mouse immune serum (m-Pdx1-IS); 3, PAA(+); and 4, PAA(−) NOD serum samples. Arrow indicates position of Pdx1 protein.

FIG. 3 shows that NOD mouse serum contains antibodies that bind to full length Pdx-1, but that fail to bind to trypsinized Pdx-1 fragments. Partial digested Pdx1 was analyzed by western blot analysis. Pdx1 protein was partially digested with trypsin at pH 7.6 25° C. for 10 min, and the digested products were separated and stained with Coomassie blue (left panel) or transferred to the membrane for blotting with two PAA(+) mouse sera (m2L and m7R, lanes 1 and 2), human PAA+ serum (H11, lane 3), or rabbit polyclonal anti-Pdx1 immune serum (lane 4).

FIG. 4 shows the expression and purification of truncated Pdx1 proteins. Full-length or truncated Pdx1 proteins with an N-terminal histidine tag were prepared as described herein, separated by SDS-PAGE, and stained with Coomassie blue dye (upper panel). Lanes 1, full-length; 2, Pdx1 (1-199); 3, Pdx1 (1-159); and 4, Pdx1 (1-119). Lower panel shows three purified proteins GST-Pdx1 (200-283) ‘p83,’ GST-Pdx1 (160-200) ‘p40,’ and GST that were separated by SDS-PAGE and stained with Coomassie blue. Lanes 5, p83; 6, p4-0; and 7, GST.

FIG. 5 shows the identification of two dominant epitope regions using a mixture of Pdx1 proteins. Four purified proteins, Pdx1 (a) and its truncated forms (b, c, and d), were mixed at equal concentrations and used for western blotting to detect autoantibodies. Each lane was loaded with 5 mg of mixed proteins, resolved by SDS-PAGE, transferred to PVDF membrane, and probed using three mouse PAA(+) sera (6L, 7R, and 4L) at a dilution of 1:1500, or six human PAA(+) sera (lanes 1-6) at a dilution of 1:200. Rabbit polyclonal anti-Pdx1 immune serum (rPdx1 IS) was used as a positive control at a dilution of 1:2000. Arrows indicate full-length Pdx1 (a) and truncated proteins (b, c, and d). Lower panel shows the structure of Pdx1 and its truncated forms and potential autoepitope regions I and II for mouse and human PAAs.

FIG. 6 shows that Pdx1 AA specifically interacts with the C-terminal portion Pdx1 protein. Upper left panel shows Coomassie blue staining of GST (lane 1), p83 (lane 2), and Pdx1 (lane 3). Right two panels are immunoblots using the same amount of proteins and blotted with two NOD mouse PAA(+) sera. Arrows indicate the positions of Pdx1 and p83. Lower left panel is a diagram of the four proteins (1, GST; 2, GST-p40; 3, GST-p83; and 4, Pdx1) used in this study. The lower right panel depicts an immunoblot using human h11 PAA(+) serum. Arrows indicate positions of corresponding proteins as indicated.

FIGS. 7A and 7B are graphs showing ELISA results following autoantibody dilution. These results indicate that the Pdx-1 autoantibody is present in mouse anti-sera at high titer, and the Pdx-1 autoantibody is highly specific for Pdx-1.

FIG. 8 shows the relationship between PAA levels and the onset of diabetes. Serum samples were collected biweekly from 5-week-old NOD mice (n=20). PAAs were detected by ELISA and expressed as OD values at 450 nM. Blood glucose levels were monitored weekly through tail vein snipping. Four representative mice (m20R, m20L, m20N, and m19L) are shown here.

FIG. 9 is a graph showing treatment of diabetes in mice by intraperitoneal administration of Pdx1 peptides. NOD mice at the age of ten weeks were treated with Pdx1 protein (IP for 4 week or 12 weeks) or PBS. N=10/each group. Percentages shown.

FIG. 10 is a graph showing that Pdx-1 treatment prevented diabetes in NOD mice.

FIG. 11 shows that Pdx-1 administration reduced NOD mice blood glucose levels to within the normal range

FIG. 12 is a graph showing that Pdx-1 treatment reverses diabetes in NOD mice.

FIG. 13 is a graph showing treatment of diabetes in mice by intraperitoneal administration of Pdx1 of Mut Pdx1 peptides. NOD mice at the age of ten weeks were treated with Pdx1 (IP for 8 weeks), Mut Pdx1 (IP for 8 weeks) or PBS. N=10/each group. Percentages shown.

FIG. 14 is a graph showing that Pdx1 and mutant Pdx1 lacking the homeobox domain were effective in reversing diabetes in NOD mice.

FIG. 15 is a graph showing treatment of diabetes in mice by subcutaneous administration of Pdx1, Mut Pdx1 or control peptides. NOD mice at the age of seven weeks were injected subcutaneously with Pdx1 protein or Mut Pdx1 protein daily for 4 weeks, and subsequently twice a week for 4 weeks. Mice in control groups were treated with GST, P120 or PBS. N=10/each group. Percentages shown.

FIG. 16 is a graph showing the blood glucose level in NOD mice treated by oral administration of Pdx1 protein or Mut Pdx1 protein. N=10/each group. Percentages shown.

FIG. 17 is a graph showing incidence of diabetes in NOD-Scid mice after adoptive transfer of splenocytes from NOD mice treated with Pdx1 protein, Mut Pdx1 protein, or PBS.

FIGS. 18A and 18B are graphs showing that splenocyte adoptive transfer from Pdx-1 treated mice or from untreated diabetic NOD mice into NOD-scid mice was sufficient to prevent or treat diabetes in NOD mice recipients. Without wishing to be bound by theory, these results indicate that Pdx-1 treatment is acting by modulating the immune response of diabetic mice. SAT mouse diabetes at 3-4 weeks.

FIGS. 19 is a graph showing that a Pdx1 peptide comprising amino acids 200-283 was most effective in stimulating lymphocyte proliferation relative to longer peptide fragments.

FIGS. 20A and 20B show that Mut Pdx1 treatment diminished insulitis and protected the insulin producing cells from loss. FIG. 20A depicts histological examination of pancreas. Pancreas in Mut Pdx1 protein treated group, eight weeks after the first GST injection, revealed islets heavily infiltrated by leukocytes. FIG. 20B shows insulitis severity scores in NOD mice treated with Mut Pdx1 (n=15) were much better than that in GST treated group. Percentages represent the number of islets of a given score over the total number of islets. Bars show the percentage of islets with the designated insulitis score.

FIG. 21 shows the functional domains of human Pdx-1.

FIG. 22 shows an alignment of Pdx-1 polypeptides from various species.

FIGS. 23A-23D are figures depicting the Luciferase Immunoprecipitation System (LIPS) for detecting Pdx1 autoantibodies (PAA). FIG. 23A is schematic depicting antibody detection using luciferase antigen fusion constructs (Luciferase Immunoprecipitation System). FIG. 23B is a schematic depicting the construction of PAA LIPS plasmids. PAA LIPS plasmids were constructed by fusing the Pdx1 gene to renilla luciferase (Ren Luc) for detection of PAA by LIPS. Plasmids were constructed for expression in both mammalian cells (pCMV) and E. coli (T7). Renilla luciferase only constructs (control) have a stop codon introduced following the coding sequence for this gene. FIG. 23C is a schematic depicting the transfection of cells with pCMV-Luc-hPdx1 plasmid and Pdx 1-Luc lysate collection. FIG. 23D is a schematic depicting RenLuc-hPdx1 immunoprecipitation with human serum and luciferase detection.

FIGS. 24A-24C are graphs depicting the detection and relationship of Pdx1 autoantibodies using LIPS and islet-cell cytoplasmic autoantibodies (ICA), glutamic acid decarboxylase autoantibodies (GADA), or insulinoma 2-associated autoantibodies (IA-2A) using LIPS and RIA. FIG. 24A is a graph depicting the detection of GADA using LIPS and RIA. A standard LIPS assay was performed with luciferase-GAD65 fusion protein lysate. FIG. 24B is a graph depicting the detection of IA-2A using LIPS and RIA. A standard LIPS assay was performed with luciferase-IA2 fusion protein lysate. FIG. 24C is a graph depicting the detection of PAA using LIPS using hPdx1 antigen. A standard LIPS assay was performed with luciferase-hPdx1 fusion protein lysate. Individual sera samples (54 donors) comprising sera determined as positive or negative for ICA, GADA, or IA-2A according to clinical data by RIA were used in all panels. In FIGS. 24A and 24B, 1 μl of serum was used and in FIG. 24C, 10 ul of serum was used for detection. In all panels, transverse line is 3SD above control mean. RLU=relative light units and are expressed as fold over control mean. Control sera comprise sera obtained from 10 normal healthy donor patients. AAb=autoantibody.

FIGS. 25A-25D depict the antigenic specificity of PAA in human sera using purified rPdx1 protein in competition assays. FIG. 25A is a graph depicting Pdx1 LIPS using Pdx1 Ab+ rabbit sera and in competition with purified rPdx1 protein. A standard LIPS assay was performed with E. coli produced renilla luciferase-mPdx1 fusion protein lysate and polyclonal Pdx1 rabbit serum (100). Purified rPdx1 protein or BSA was incubated overnight at indicated concentrations. FIG. 25B is a graph depicting Pdx1 LIPS using LS T1D PAA+ sera and in competition with purified rPdx1 protein. A standard LIPS assay was performed with E. coli produced renilla luciferase-mPdx1 fusion protein lysate and LS T1D serum (100). Purified rPdx1 protein or BSA was incubated overnight at indicated concentrations. FIG. 25C is a graph depicting LIPS in competition with Pdx1 and Pdx1 c-terminal truncated peptides using immune serum or autoimmune serum. A standard LIPS assay was performed with E. coli produced renilla luciferase-mPdx1 fusion protein lysate and either Pdx1 Ab+ rabbit sera (100) or LS T1D serum (100). In addition 10 μg/μl purified full length rPdx1 protein (283aa), purified rPdx1 with various C-terminal truncations (first 200aa, 160aa, or 120aa), or BSA was incubated overnight. aa=amino acids. FIG. 25D is a graph depicting LIPS assay using luciferase-Pdx1 fusion protein compared to luciferase-only antigen. A standard LIPS assay was performed with either luciferase-only protein lysate or luciferase-hPdx1 fusion protein lysate as indicated. The ctrl serum (10 ul) is negative for PAA and sera A-C (10 ul) are high-signal PAA-positive sera. For all figures, RLU=relative light units and are expressed as fold over control.

FIGS. 26A-26E depict PAA detection using LIPS in T1D (recent onset and long standing), Systemic Lupus Erythematosus, Rheumatoid arthritis, and pancreatic cancer. FIG. 26A is a graph depicting PAA detection using LIPS in sera from recent onset T1D subjects. FIG. 26B is a graph depicting PAA detection using LIPS in sera from long standing T1D subjects. FIG. 26C is a graph depicting PAA detection using LIPS in sera from subjects having Systemic Lupus Erythematosus. FIG. 26D is a graph depicting PAA detection using LIPS in sera from subjects having rheumatoid arthritis. FIG. 26E is a graph depicting PAA detection using LIPS in sera from subjects having cancer. In all panels, a standard LIPS assay was performed with luciferase-hPdx1 fusion protein lysate. 10 μl of indicated sera was used. In all panels, transverse line is 3SD above control mean. RLU=relative light units and are expressed as fold over control mean. Control sera comprise sera obtained from 10 normal healthy donor patients.

### DETAILED DESCRIPTION OF THE INVENTION

The invention features compositions and methods that are useful for diabetes diagnosis treatment, and prevention. The invention is based, at least in part, on the following discoveries: first, that anti-Pdx1 autoantibodies (Pdx1 AA) were detected in non-obese diabetic (NOD) mice using ELISA, Western blotting, and immunoprecipitation of [³⁵S]-labeled Pdx1; second, that treatment of mice with full length or truncated Pdx1 proteins prevented or delayed diabetes onset; third, that treatment of mice with Pdx-1 protein lacking the homeodomain was sufficient to reverse diabetes in NOD mice; and finally, that Pdx-1 is likely acting by modulating the autoimmune response of the NOD mice.

Pdx1 is a key transcription factor involved in the regulation of insulin gene expression that is expressed at high levels in the β-cells of the pancreatic islets. As reported herein, to determine whether Pdx1 is a target of anti-islet autoimmunity in Type 1 diabetes, anti-Pdx1 autoantibodies (Pdx1 AA) were detected in non-obese diabetic (NOD) mice using ELISA, Western blotting, and immunoprecipitation of [³⁵S]-labeled Pdx1. Pdx1 AA were detected as early as 5 weeks of age in NOD mice. This is at least 6-8 weeks before the onset of clinically overt diabetes in diabetes-prone female mice. Levels of Pdx1 autoantibodies peaked at ˜9-16 weeks of age, then declined or disappeared after diabetes onset. The anti-Pdx1 AA were not detected in the sera of BALB/c or NOD-Scid mice. The titers of anti-Pdx1 AA in NOD mouse sera ranged from 1/1000 to 1/50,000 by ELISA. The specificity of anti-Pdx1 AA was determined by Western blotting using a series of truncated recombinant Pdx1 proteins. The immunodominant epitope was localized to the Pdx1 C-terminus (terminal 83 amino acid residues). Using [³H]-thymidine incorporation, the p83 fragment of Pdx1 specifically stimulated the proliferation of splenic T-cells from NOD mice with recent onset diabetes (7-fold increase over control). Sera from some patients with type 1 diabetes contains Pdx1 AA recognizing epitopes different from those bound by NOD sera. The dominant epitope recognized by human Pdx1 autoantibodies appears to be localized between amino acids 156-200. Treatment of prediabetic NOD mice at 11 weeks with non-functional, mutant Pdx1 protein either prevented or delayed the onset of clinical diabetes. The presence of anti-Pdx1 autoantibodies in prediabetic NOD mice and the prevention of diabetes using mutant Pdx1 indicates that immune responses against Pdx1 likely plays a part in Type 1 diabetes (T1D) pathology.

In sum, as reported below, Pdx1 is an autoantigen that plays an important role in the pathogenesis of type 1 diabetes. Anti-Pdx1 autoantibodies (AA) are an important marker for prediction of the onset of T1D, and confirmation of the diagnosis of T1D. Accordingly, the invention provides diagnostic compositions and methods. The epitopes for autoantibody and T-cell response have been localized within a certain region of the Pdx1 polypeptide. Specifically, mouse Pdx-1 autoantibodies bind to Pdx-1 amino acids 200-283 and human Pdx-1 autoantibodies binds to Pdx-1 amino acids 150-200. Pdx-1 treatment of NOD mice modulated their immune response and delayed diabetes onset in a splenocyte adaptive transfer experiment. Importantly, treatment of NOD mice with mutant Pdx1 lacking the homeodomain that mediates DNA binding prevented or delayed the onset of T1D in Mice. Accordingly, the invention further provides Pdx1 peptides that may be used as therapeutic reagents for the prevention or treatment of diabetes. For example, a Pdx1 peptide may be particularly useful for the prevention of diabetes in patients in a prediabetic stage, or for the treatment of subjects with diabetes, particularly those having the recent onset of T1D.

Accordingly, the invention provides Pdx-1 polypeptides or peptides having immunomodulatory activity. Therapeutic compositions comprising Pdx-1 polypeptides or peptides are administered to a subject to treat or prevent pre-diabetes or diabetes. Without wishing to be bound by theory, the administration of a Pdx-1 polypeptide or fragment thereof likely acts to reduce a Pdx-1-specific immune response, to induce immunotolerance, to disrupt binding of a Pdx-1 antibody to an endogenous Pdx-1 polypeptide, to reduce the effects of Pdx-1 autoantibodies (i.e., antibodies that specifically bind Pdx-1). The present invention provides for the detection and monitoring of Pdx-1 autoantibodies for early diagnosis and management of hyperglycemia, pre-diabetes or diabetes, or a predisposition thereto.

### Diagnostics

Accordingly, the present invention provides diagnostic assays for the detection of pre-diabetes, diabetes, or the propensity to develop such conditions. Levels of Pdx-1 specific antibodies are measured in a subject sample (e.g., blood, serum, plasma, urine, saliva). Standard methods may be used to measure levels of Pdx-1 specific antibodies in any bodily fluid, including, but not limited to, blood, urine, serum, plasma, or saliva. Such methods include immunoassay, ELISA, Western blotting using a Pdx-1 peptide or polypeptide, reporter assays, e.g., a luciferase reporter assay using a Pdx-1 fused to luciferase, and radioimmunoas say. In general, Pdx-1 autoantibodies are not detectable in healthy subjects (i.e., those who do not have and/or who will not develop diabetes). The presence of Pdx-1 specific antibodies in a subject sample is an indicator of pre-diabetes, diabetes, or a propensity to develop such conditions. Similarly, failure to detect (or detection of a nominal amount) Pdx-1 autoantibodies in a subject sample is indicative that the subject does not have, or does not have a propensity to develop pre-diabetes or diabetes. In one embodiment, any detectable amount of Pdx-1 specific antibody is indicative of pre-diabetes or diabetes in a subject. In another embodiment, the detection of increased serum levels of Pdx-1 specific antibodies (relative to levels detected in a normal control sample) is considered a positive indicator of pre-diabetes or diabetes or the propensity to develop such conditions.

In one embodiment, the level of Pdx-1 specific antibodies is measured at least two different times and an alteration in the levels as compared to normal reference levels over time is used as an indicator of pre-diabetes, diabetes, or the propensity to develop such conditions. The level of Pdx-1 specific antibodies in the bodily fluids of a subject having pre-diabetes or diabetes may be altered by as little as 10%, 20%, 30%, or 40%, or by as much as 50%, 60%, 70%, 80%, or 90% or more relative to the level of Pdx-1 specific antibodies in a normal control. In one embodiment, a subject sample of a bodily fluid (e.g., blood, urine, plasma, serum) is collected prior to the onset of pre-diabetic or diabetic symptoms. In another example, the sample can be a tissue or cell collected prior to the onset of pre-diabetic or diabetic symptoms.

The diagnostic methods described herein can be used individually or in combination with any other diagnostic method described herein for a more accurate diagnosis of the presence, severity, or estimated time of onset of pre-diabetes or diabetes. For example, if desired, pre-diabetes or diabetes diagnosis is made by assaying a subject sample for anti-Pdx-1 antibodies, as well as for antibodies against other autoantigens associated with pre-diabetes or diabetes. Such autoantigens include, but are not limited to, GAD65, IA-2 (also known as ICA512), and insulin. In addition, the diagnostic methods described herein can be used in combination with any other diagnostic methods determined to be useful for the accurate diagnosis of the presence of, severity of, or estimated time of onset of pre-diabetes or diabetes. The diagnostic methods described herein can also be used to monitor and manage pre-diabetes or diabetes.

### Diagnostic Kits

The invention provides for a pre-diabetes or diabetes diagnostic kit. In one embodiment, a diagnostic kit includes a means for detecting binding between Pdx-1 specific antibodies and a Pdx-1 polypeptide. For example, a Pdx-1 polypeptide-antibody interaction can be detected in any standard immunoassay. A conventional ELISA is a common, art-known method for detecting antibody-substrate interaction and can be provided with a kit of the invention. Pdx-1 antibodies can be detected in virtually any bodily fluid including, but not limited to blood, serum, plasma, urine, or saliva. A kit that detects the presence of Pdx-1 specific antibodies or detects an increase in the level of Pdx-1 specific antibodies relative to a reference, such as the level present in a normal control, is useful as a diagnostic kit. If desired, the kit can detect one, two, or three additional autoantigens associated with diabetes (e.g., GAD65, IA-2, and insulin). Desirably, the kit will contain instructions for the use of the kit. In one example, the kit contains instructions for the use of the kit for the diagnosis of pre-diabetes or diabetes, or the propensity to develop pre-diabetes or diabetes. In yet another example, the kit contains instructions for the use of the kit to monitor therapeutic treatment or dosage regimens.

### Subject Monitoring

The disease state or treatment of a subject having pre-diabetes or diabetes, or a propensity to develop such conditions can be monitored using the methods and compositions of the invention. In one embodiment, the expression of Pdx-1 specific antibodies present in a bodily fluid, such as blood, serum, plasma, urine, or saliva, is monitored. Such monitoring may be useful, for example, in assessing the efficacy of a particular drug in a subject or in assessing disease progression. If desired, the diagnostic methods of the invention can be used in combination with other conventional diagnostics for the detection of diabetes, such diagnostics include, but are not limited to, the detection of increases blood glucose levels, the detection of a reduction in insulin levels, the detection of a reduction in the number or viability of pancreatic beta cells, the detection of autoantigens (e.g., GAD65, IA-2, and insulin). Diagnostic tests for diabetes include, for example, fasting plasma glucose (FPG) test, oral glucose tolerance test, and random plasma glucose test.

Therapeutics that decrease the expression of Pdx-1 specific antibodies are taken as particularly useful in the invention. The diagnostic methods described herein can also be used to determine the dosages of therapeutic compounds. In one example, a therapeutic compound is administered and the level of Pdx-1 specific antibodies is determined during the course of therapy. If the level of Pdx-1 specific antibodies is reduced (e.g., by at least about 5, 10, 20, 30, 40, 50, 75, or 100%) by treatment, then the therapeutic dosage is considered to be an effective dosage.

### Therapeutics

The present invention also features methods for treating or preventing pre-diabetes or diabetes in a subject. In one embodiment, a therapeutic composition of the invention (e.g., a composition comprising a Pdx-1 polypeptide or fragment thereof) is administered to a subject prior to the onset of diabetes, to a subject identified as having pre-diabetes or diabetes, or to a subject having pre-diabetic or diabetic symptoms. For example, the invention provides for the administration of full length or truncated Pdx-1 polypeptides to a subject identified as having Pdx-1 autoantibodies. Desirably, the Pdx-1 polypeptides or peptides are capable of binding a Pdx-1 specific antibody (e.g., an autoantibody present in a subject identified as having or having a propensity to develop diabetes or pre-diabetes), of reducing a Pdx-1 specific immune response in a subject, of inducing immunological tolerance, or of otherwise treating or preventing pre-diabetes or diabetes. Techniques and dosages for administration vary depending on the type of compound (e.g., peptide, polypeptide, chemical compound, or nucleic acid vector) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral or local by direct injection. The composition can be in the form of a pill, tablet, capsule, liquid, or sustained release tablet for oral administration; or a liquid for intravenous, subcutaneous or parenteral administration; or a polymer or other sustained release vehicle for local administration.

Methods well known in the art for making formulations are found, for example, in “Remington: The Science and Practice of Pharmacy” (20th ed., ed. A. R. Gennaro AR., 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the agent depends on various clinical factors including the overall health of the subject and the severity of the symptoms of pre-diabetes or diabetes. In general, once diabetes, pre-diabetes or a propensity to develop diabetes is detected, daily, weekly, or continuous infusion of a purified peptide is used to treat or prevent pre-diabetes or diabetes. Treatment can be continued for a period of time. For example, treatment may be administered indefinitely for as long as the patient shows a propensity to develop pre-diabetes or diabetes (e.g., for days, months, or years). Dosages vary depending on the peptide and the severity of the condition and are titrated to achieve a steady-state blood serum concentration ranging from 1 to 500 ng/mL Pdx-1 polypeptide or peptide, preferably 1 to 100 ng/mL, more preferably 5 to 50 ng/mL and most preferably 5 to 10 ng/mL. In one embodiment, a Pdx-1 imunnological composition is administered once or twice daily, for example, by subcutaneous injection, where the amount administered is 10 μg, 100 μg, 1000 μg, 5 mg, 10 mg, 25 mg, 50 mg, 100 mg, 200 mg, 250 mg, 500 mg, or 1000 mg. If desired, the Pdx-1 therapeutic is administered one, two, three, four, or five times a week. Therapeutic regimens may be continued for 3 or 4 weeks or for 3, 6, 9 or 12 months, or indefinitely.

Recombinant Pdx-1 polypeptide and fragments thereof are administered as prophylactic agents in subjects having a propensity to develop diabetes, or can be used as therapeutic agents for treating subjects suffering from diabetes. Accordingly, the invention provides immunomodulatory compositions comprising a Pdx-1 polypeptide or fragment thereof. Immunogenic compositions or vaccines of the invention can be administered to humans or in veterinary contexts, e.g., in the vaccination of domestic pets (e.g., cats, dogs, and birds) or livestock (e.g., horses, sheep, cattle, pigs, birds, and goats). Further, the immunogenic compositions or vaccines of the invention can include other autoantigens associated with diabetes (e.g., GAD65, IA-2, and insulin).

Formulation of the polypeptides of the invention can be carried out using methods that are standard in the art. Numerous pharmaceutically acceptable solutions for use in the preparation of immunogenic compositions and vaccines are well known and can readily be adapted for use in the present invention by those of skill in this art (see, e.g., Remington's Pharmaceutical Sciences (18.sup.th edition), ed. A. Gennaro, 1990, Mack Publishing Co., Easton, Pa.). For example, the polypeptides can be diluted in a physiologically acceptable solution, such as sterile saline or sterile buffered saline. In another example, the polypeptides can be administered and formulated, for example, as a fluid harvested from cell cultures secreting the recombinant polypeptides.

The immunogenic compositions and vaccines of the invention can be administered using methods that are well known in the art, and appropriate amounts of the immunogenic compositions and vaccines to be administered can readily be determined by those of skill in the art. What is determined to be an appropriate amount of polypeptides to administer can be determined by consideration of factors such as, e.g., the size and general health of the subject to whom the polypeptide is to be administered. For example, the polypeptides of the invention can be formulated as sterile aqueous solutions containing between 1 μg and 1 mg in a dose volume of 0.1 to 1.0 ml, to be administered by, for example, intramuscular, subcutaneous, or intradermal routes. In various embodiments, the immunogenic composition or vaccine comprises about 1, 5, 10, 25, 50, 100, 200, 300, 500, 750, or 1000 μg. Further, the immunogenic compositions and vaccines of the invention can be administered in a single dose or, optionally, administration can involve the use of a priming dose followed by one or more booster doses that are administered, e.g., 2-6 months later, as determined to be appropriate by those of skill in the art. In other embodiments, the immunogenic compositions and vaccines are injected at daily, weekly, or monthly intervals to suppress an immune response or induce immunological tolerance.

Optionally, adjuvants that are known to those skilled in the art can be used in the administration of the polypeptides of the invention. Adjuvants that can be used to enhance the immunogenicity of the polypeptides include, for example, liposomal formulations, synthetic adjuvants, such as (e.g., QS21), muramyl dipeptide, monophosphoryl lipid A, or polyphosphazine. The invention also includes nucleic acid molecules (e.g., RNA or DNA (e.g., cDNA) molecules) that encode the polypeptides of the invention as described herein, or the complements thereof. These nucleic acid molecules can be used, for example, in methods of manufacturing the polypeptides of the invention. In such methods, a nucleic acid molecule encoding the polypeptide is introduced into cells in which the polypeptides can be produced and from which (or the supernatants of which) the polypeptides can then be purified. These methods can further include polypeptide purification steps, as is known in the art.

### Purified Proteins

As described herein, administration of full length or truncated Pdx-1 polypeptides are useful for the treatment or prevention of diabetes. Purified Pdx-1 or Pdx-1-like proteins include any protein with an amino acid sequence that is homologous, more desirably, substantially identical to the amino acid sequence of Pdx-1. Desirably, Pdx-1 polypeptides, peptides, or analogs thereof are capable of binding a Pdx-1 specific antibody (e.g., an autoantibody present in a subject identified as having or having a propensity to develop diabetes or pre-diabetes), of reducing a Pdx-1 specific immune response in a subject, of inducing immunological tolerance, or of otherwise treating or preventing pre-diabetes or diabetes. Preferred Pdx-1 peptides comprise at least 5, 10, 20, 30, 40, 50, 60, 70, 80, 90, 100, 110, 120, or 150 amino acids from the C-terminus of a Pdx-1 polypeptide. In one embodiment, a human Pdx-1 peptide comprises at least about amino acids 150-200 or 200-283/284. If desired the Pdx-1 peptide includes 5, 10, 20, 30, 40, or 50 amino acids flanking the carboxy or amino terminus of the Pdx-1 peptide (e.g., flanking Pdx-1 amino acids 150-200 or 200-283/284). In another embodiment, the Pdx-1 peptide comprises or consists essentially of at least about amino acids 200-283/284.

### Recombinant Polypeptide Expression

The invention provides Pdx-1 polypeptides and fragments thereof. Pdx-1 polypeptides and peptides are expressed as recombinant polypeptides using virtually any method known to the skilled artisan. Typically, recombinant polypeptides are produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

The present invention provides methods of treating disease and/or disorders or symptoms thereof which comprise administering a therapeutically effective amount of a pharmaceutical composition comprising an agent (e.g., Pdx-1 polypeptide or peptide) of the formulae herein to a subject (e.g., a mammal, such as a human). Thus, one embodiment is a method of treating a subject suffering from or susceptible to a disease or disorder or symptom thereof. The method includes the step of administering to the mammal a therapeutic amount of an agent herein sufficient to treat the disease or disorder or symptom thereof, under conditions such that the disease or disorder is treated.

The methods herein include administering to the subject (including a subject identified as in need of such treatment) an effective amount of an agent described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of an agent herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which hyperglycemia, diabetes, or a Pdx-1-specific immune response may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with diabetes, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pdx-1 Polypeptides and Analogs

Also included in the invention are Pdx-1 polypeptides or fragments thereof that are modified in ways that enhance their ability to bind a Pdx-1 specific antibody (e.g., an autoantibody present in a subject identified as having or having a propensity to develop diabetes or pre-diabetes), that reduce a Pdx-1 specific immune response in a subject, that induce immunological tolerance, or that otherwise treat or prevent pre-diabetes or diabetes in a subject having a propensity to develop such conditions.

The invention provides methods for optimizing a Pdx-1 amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 50%, 75%, 80%, 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. In one embodiment, a fragment may include about 35, 40, 45, 50, 60, 65, 70, 75 or 100 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein Pdx-1 analogs have a chemical structure designed to mimic the protein's functional activity. Such analogs are administered according to methods of the invention. Pdx-1 protein analogs may exceed the physiological activity of the original polypeptide. Methods of analog design are well known in the art, and synthesis of analogs can be carried out according to such methods by modifying the chemical structures such that the resultant analogs increase the activity of a reference Pdx-1 polypeptide. These chemical modifications include, but are not limited to, substituting alternative R groups and varying the degree of saturation at specific carbon atoms of a reference Pdx-1 polypeptide. Preferably, the Pdx-1 analogs are relatively resistant to in vivo degradation, resulting in a more prolonged therapeutic effect upon administration. Assays for measuring functional activity include, but are not limited to, those described in the Examples below.

### Therapeutic Nucleic Acids

Delivery of a nucleic acid molecule (DNA or RNA) that encodes a Pdx-1 polypeptide or fragment thereof is also useful for the treatment or prevention of pre-diabetes or diabetes. In the present invention the nucleic acid may be any nucleic acid (DNA or RNA) including genomic DNA, cDNA, and mRNA, encoding a Pdx-1 peptide or polypeptide. The nucleic acids encoding the desired protein may be obtained using routine procedures in the art, e.g. recombinant DNA, PCR amplification.

To simplify the manipulation and handling of the nucleic acid encoding the Pdx-1 polypeptide, the nucleic acid is preferably inserted into a cassette where it is operably linked to a promoter. The promoter must be capable of driving expression of the Pdx-1 protein in the desired target host cell. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum. Gene Ther. 4:151-159, 1993) and mouse mammary tumor virus (MMTV) promoters may also be used. Certain proteins can be expressed using their native promoter. Other elements that can enhance expression can also be included (e.g., enhancers or a system that results in high levels of expression such as a tat gene and tar element). The recombinant vector can be a plasmid vector, such as pUC118, pBR322, or other known plasmid vectors, that includes, for example, an E. coli origin of replication (see, Sambrook, et al., Molecular Cloning: A Laboratory Manual, Cold Spring Harbor Laboratory press, 1989). The plasmid vector may also include a selectable marker such as the B lactamase gene for ampicillin resistance, provided that the marker polypeptide does not adversely affect the metabolism of the organism being treated.

The nucleic acid can be introduced into the cells by any means appropriate for the vector employed. Many such methods are well known in the art (Sambrook et al., supra, and Watson et al., “Recombinant DNA”, Chapter 12, 2d edition, Scientific American Books, 1992). Recombinant vectors can be transferred by methods such as calcium phosphate precipitation, electroporation, liposome-mediated transfection, gene gun, microinjection, viral capsid-mediated transfer, polybrene-mediated transfer, or protoplast fusion. For a review of the procedures for liposome preparation, targeting and delivery of contents, see Mannino and Gould-Fogerite, (Bio Techniques, 6:682-690, 1988), Felgner and Holm, (Bethesda Res. Lab. Focus, 11:21, 1989) and Maurer (Bethesda Res. Lab. Focus, 11:25, 1989). In one embodiment, transfer of the recombinant vector (either plasmid vector or viral vectors) is accomplished through intravenous delivery.

Gene delivery using adenoviral vectors or adeno-associated vectors (AAV) can also be used. Adenoviruses are present in a large number of animal species, are not very pathogenic, and can replicate equally well in dividing and quiescent cells. As a general rule, adenoviruses used for gene delivery are lacking one or more genes required for viral replication. Replication-defective recombinant adenoviral vectors used for the delivery of a Pdx-1 polypeptide or peptide can be produced in accordance with art-known techniques (see Quantin et al., Proc. Natl. Acad. Sci. USA, 89:2581-2584, 1992; Stratford-Perricadet et al., J. Clin. Invest., 90:626-630, 1992; and Rosenfeld et al., Cell, 68:143-155, 1992).

A variety of methods are available for transfection, or introduction, of nucleic acid molecules into mammalian cells. For example, there are several commercially available transfection reagents including but not limited to: TranslT-TKO (Mirus, Cat. # MIR 2150), TRANSMESSENGER (Qiagen, Cat. #301525), and OLIGOFECTAMINE (Invitrogen, Cat. # MIR 12252-011). Protocols for each transfection reagent are available from the manufacturer. Once transferred, the nucleic acid is expressed by the cells at the site of injury for a period of time sufficient to increase blood serum levels of Pdx-1. Because the vectors containing the nucleic acid are not incorporated into the genome of the cells, expression of the protein of interest takes place for only a limited time. Typically, the protein is expressed at therapeutic levels for about two days to several weeks, preferably for about one to two weeks. Re-application of the DNA can be utilized to provide additional periods of expression of the therapeutic protein.

### Assays for Pdx-1 Gene and Protein Expression

The following methods can be used to evaluate Pdx-1 protein or gene expression and determine efficacy for any of the above-mentioned methods for increasing Pdx-1 protein levels. Blood serum from the subject is measured for levels of Pdx-1 polypeptide or peptides. Methods used to measure serum levels of proteins include ELISA, western blotting, or immunoassays. The present invention provides Pdx-1 polypeptide or peptides that bind a Pdx-1 antibody or that otherwise modulate a Pdx-1 specific immune response.

### Combination Therapies

Optionally, a pre-diabetes or diabetes therapeutic may be administered in combination with any other standard pre-diabetes or diabetes therapy; such methods are known to the skilled artisan. For example, a pre-diabetes or diabetes therapeutic of the invention may be administered in combination with insulin. In other embodiments, a Pdx-1 polypeptide or peptide of the invention is administered in combination with other autoantigens (e.g., GAD65, IA-2, and insulin) to suppress or reduce an immune response, to induce immunological tolerance, or to otherwise treat, prevent, delay, or ameliorate a symptom of diabetes. In some embodiments, the methods of the invention reduce or delay the need for insulin therapy or otherwise reduce the severity of diabetes or a symptom thereof.

### Screening Assays

As discussed above, Pdx-1 specific antibodies are detectable in a subject having pre-diabetes, diabetes, or a propensity to develop such conditions. Based on these discoveries, compositions of the invention are useful for the high-throughput low-cost screening of candidate agents to identify those that reduce an immune response (e.g., that reduces expression of a Pdx-1 specific antibody), that induce immunological tolerance, that inhibit Pdx-1 polypeptide/Pdx-1 antibody binding, or that otherwise treats or prevents diabetes in a subject. Agents identified according to the methods of the invention are useful for the treatment or prevention of pre-diabetes or diabetes.

Any number of methods are available for carrying out screening assays to identify candidate compounds useful in the methods of the invention. In one example, candidate compounds are tested in NOD mice between the ages of 5-15 weeks, or are added at varying concentrations to the culture medium of cultured cells expressing Pdx-1 specific antibodies. The effect of candidate compounds is then measured, for example, at the level of antibody production using standard immunological techniques, such as Western blotting or immunoprecipitation with Pdx-1 polypeptide. For example, immunoassays may be used to detect or monitor the expression of Pdx-1 specific antibodies in an organism. The presence of Pdx-1 specific antibodies may be measured in any standard immunoassay format (e.g., ELISA, western blot, or radioimmunoassay). A compound that reduces a Pdx-1 specific immune response is considered particularly useful. Again, such a molecule may be used, for example, as a therapeutic to delay, ameliorate, or treat pre-diabetes or diabetes, or the symptoms of pre-diabetes or diabetes, in a subject.

In another example, agents may be screened for those that disrupt binding between a Pdx-1 polypeptide and a Pdx-1 specific antibody. The efficacy of such an agent is dependent upon its ability to interact with Pdx-1 specific antibodies or a functional equivalent thereof to reduce binding to an endogenous Pdx-1 polypeptide. Such an interaction can be readily assayed using any number of standard binding techniques and functional assays (e.g., those described herein or in Ausubel et al., supra). In one embodiment, a candidate agent may be tested in vitro for its ability to bind Pdx-1 specific antibodies.

In one example, a candidate agent that binds to a Pdx-1 specific antibody may be identified using a chromatography-based technique. In one embodiment, the agent is a recombinant Pdx-1 peptide or analogs thereof. Such peptides are expressed and purified by standard techniques from cells engineered to express them. The Pdx-1 peptides or analogs thereof are then passed over a column or other substrate containing immobilized Pdx-1 specific antibodies. Peptides that bind to the Pdx-1 antibodies are immobilized on the column (or substrate). To isolate the peptides, the column is washed to remove non-specifically bound molecules, and peptides of interest are released from the column, collected, and identified. Peptides isolated by this approach are used, for example, as therapeutics to treat pre-diabetes or diabetes in a human subject. Peptides that are identified as binding to a Pdx-1 specific antibody of the invention with an affinity constant less than or equal to 10 mM are considered particularly useful in the invention. Alternatively, any protein interaction detection system may be utilized to identify peptides that bind to a Pdx-1 specific antibody of the invention.

Candidate agents include organic molecules, peptides, peptide mimetics, polypeptides, nucleic acids, and antibodies that bind to Pdx-1 specific antibodies. Such agents may also be used in the discovery and development of a therapeutic compound for the treatment of pre-diabetes or diabetes.

### Test Compounds and Extracts

In general, agents that reduce an immune response, reduce Pdx-1 antibody expression, reduce Pdx-1 antibody binding to a Pdx-1 polypeptide, or induce immunological tolerance are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their immunomodulatory activity should be employed whenever possible.

When a crude extract is found to decrease Pdx-1 antibody expression, reduce Pdx-1 antibody binding to a Pdx-1 polypeptide, or reduce a Pdx-1-specific immune response further fractionation of the positive lead extract is necessary to isolate chemical constituents responsible for the observed effect. Methods of fractionation and purification of such heterogeneous extracts are known in the art. If desired, agents shown to be useful as therapeutics for the treatment of a human pre-diabetes or diabetes are chemically modified according to methods known in the art.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Pdx-1 Autoantibodies Present in NOD Mice

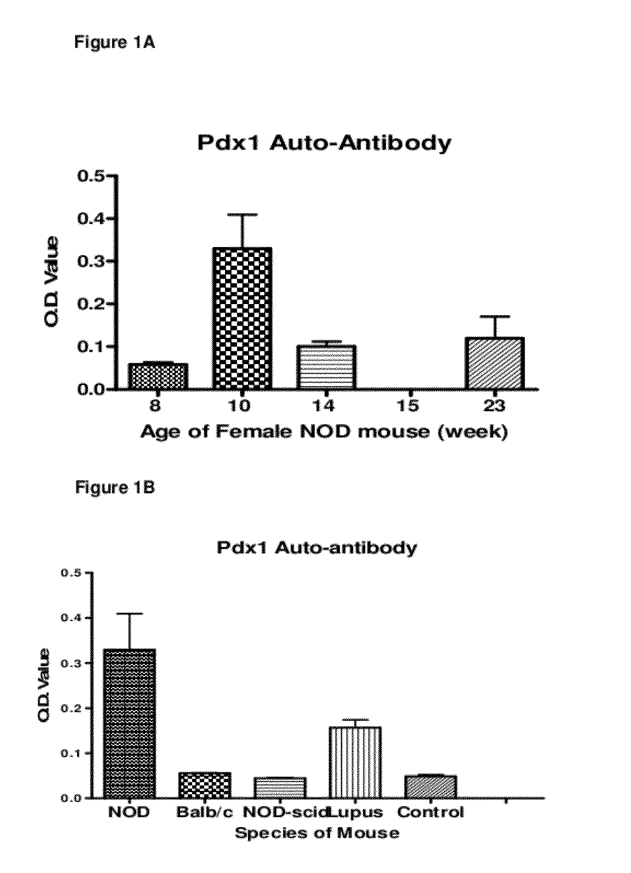

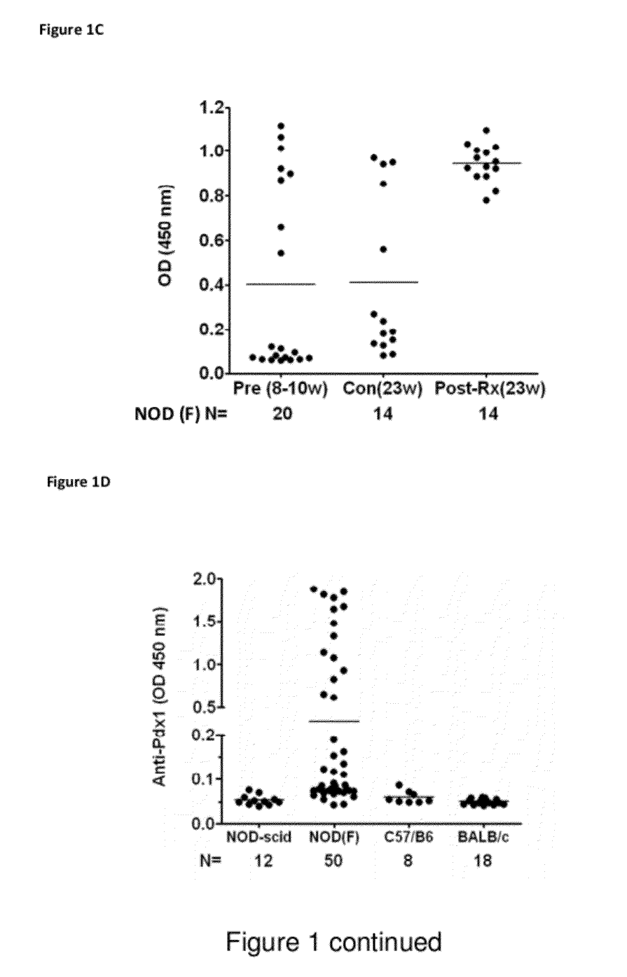

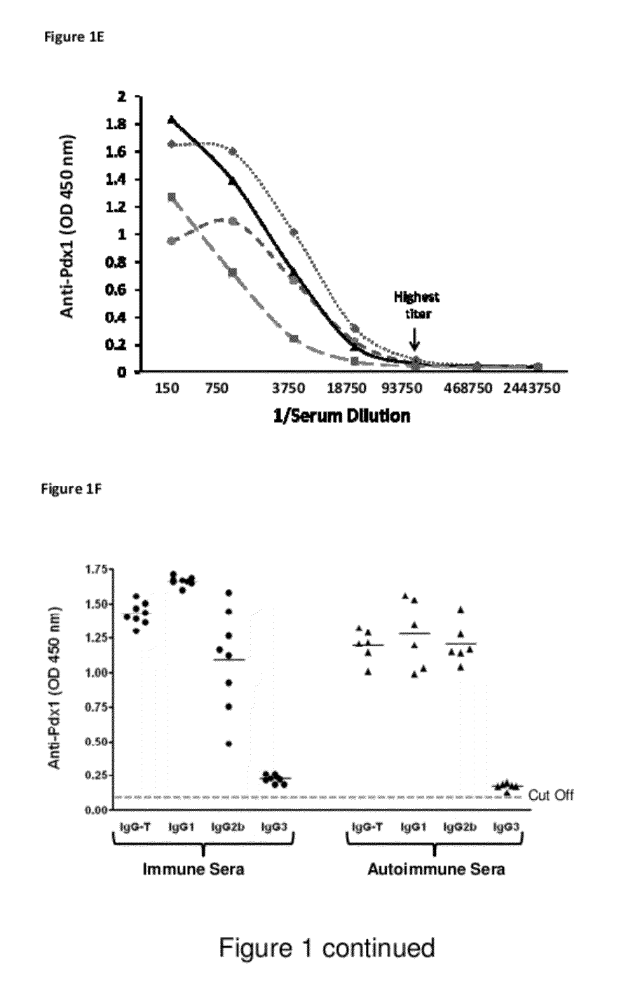

An ELISA assay was developed to detect Pdx-1 autoantibodies. The Pdx1 ELISA assay was developed and validated using purified recombinant rat Pdx1 protein and polyclonal antibody raised in rabbits using Pdx1 protein (FIGS. 1A and 1B). Increased levels of Pdx-1 autoantibodies were identified in 10 week old NOD mice. This is prior to the onset of diabetes in NOD mice, which typically occurs between 12 and 14 weeks. Mouse sera from different species (NOD-female, Balb/c, NOD-scid, C57, B6) were collected and tested for the presence of anti-Pdx1 autoantibody (Pdx1 AA) by ELISA. The results revealed that more than 90% of the serum samples in pre-diabetic NOD female mice had high levels of Pdx1 AA (FIG. 1B). Interestingly, Pdx1 autoantibodies were not detected in most other mice, with the exception of a transgenic mouse model of lupus, which is an autoimmune disorder.

Serum samples collected from pretreated, post-rPdx1-treated, and saline-treated (control) female NOD mice were tested for the presence of antiPdx1 antibodies (FIG. 1C). As expected, serum samples collected from 23-week-old mice treated with Pdx1 protein for 12 weeks showed strong immunoreactivity to Pdx1 antigen, consistent with a humoral immune response to rPdx1. Surprisingly, some serum samples from prediabetic (pre-treatment) and control mice were also strongly positive. This result raised the possibility that Pdx1 might be an islet cell autoantigen in NOD mice, leading to the production of Pdx1 autoantibodies (PAAs).

To test this hypothesis, serum samples from several mouse strains including C57BL/6, BALB/c, and nondiabetic congenic NOD-scid mice were examined for the presence of PAAs. FIG. 1D shows that 54% of prediabetic female NODmice had PAAs detectable at the screening dilution of 1:30, whereas no reactivity was seen in other mouse strains. These results indicated that PAAs were produced selectively by prediabetic NOD mice. Serial dilution of selected positive NOD mouse sera at age 10 weeks showed that the titers of these PAAs were as high as 1:93,750 (FIG. 1E). Induced anti-Pdx1 antibodies in immunized NOD mice and spontaneous antibodies in un-immunized PAA-positive (PAA+) NOD mice consisted of various isotypes (FIG. 1F). IgG1 and IgG2b anti-Pdx1 antibodies predominated in the response to immunization as well as in the spontaneous response, whereas IgG3 PAAs were less abundant (FIG. 1F). The levels of anti-Pdx1 IgG2b antibodies were considerably more variable in immunized mice than in the mice with spontaneous PAAs. Anti-Pdx1 IgG2c antibody levels could not be accurately measured (e.g., using a commercial kit), because NOD mice that express the Igh1b allele express IgG2c instead of IgG2a. Thus, the results demonstrated high levels of Pdx1 AA were associated with diabetes in mice.

### Example 2

### NOD Mouse Serum Binds Pdx-1 in Western Blot

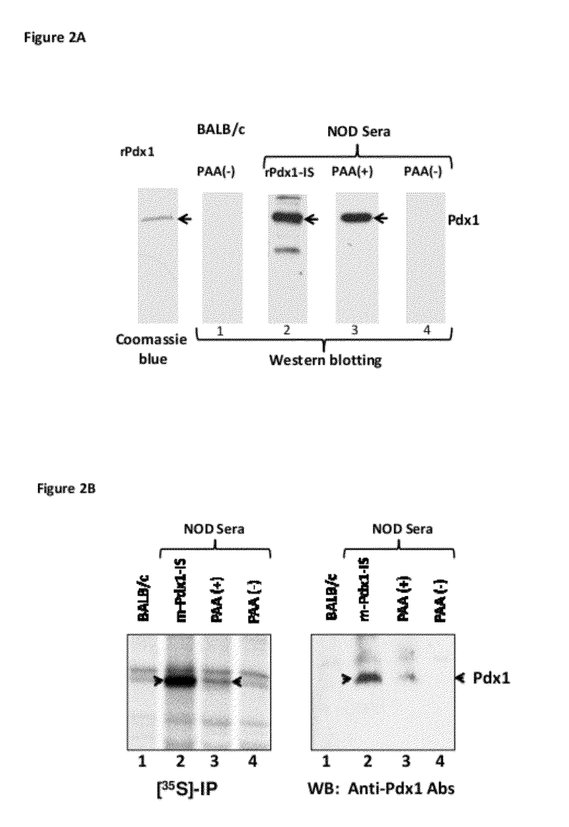

The increase in Pdx-1 autoantibody levels observed using ELISA were confirmed by Western blot. To further establish the existence of PAAs in NOD mice, selected PAA+ or PAA− negative NOD sera, negative control (BALB/c) serum, and positive control rabbit polyclonal immune serum (rPdx1-IS) were used for western blotting using purified rat Pdx1 as antigen (FIG. 2A). ELISA-PAA+ NOD mouse sera detected a single band at 46 kDa (FIG. 2A, lane 3), whereas ELISA-negative serum samples from BALB/c (FIG. 2A, lane 1) or NOD (FIG. 2A, lane 4) mice did not. In contrast, the immune serum (FIG. 2A, lane 2) showed a major band at 46 kDa as well as several minor bands, possibly due to antibodies against minor proteins contaminating the Pdx1 antigen preparation used for immunization and/or degradation fragments of Pdx1. In addition, autoantibody specificity was verified by immunoprecipitating [³⁵S]-labeled native Pdx1 from rat insulinoma INS-1 cell extracts. A band at 46 kDa that was immunoprecipitated by both NOD mouse Pdx1-treated serum (m-Pdx1-IS) (FIG. 2A, lane 2) and the ELISA-positive PAA serum (FIG. 2A, lane 3), whereas no clear band was detected in PAA-negative or control sera (FIG. 2B, left panel). The identity of this immunoprecipitated protein band was confirmed by western blotting using rabbit anti-Pdx1 polyclonal antibodies to probe the membrane (FIG. 2B, right panel). Thus, PAAs could be detected by western blotting and IP in addition to ELISA.

Serum from NOD mouse detected one clear Pdx1 band by Western blotting. Similar findings were also observed when rPdx-1 Western blots were probed with serum samples collected from human subjects with type 1 diabetes. These results indicate that Pdx1 is an autoantigen. Pdx1 protein may be released from the pancreas during islet beta cell destruction caused by ongoing autoimmunity in NOD mice.

### Example 3

### Pdx-1 Autoantibodies Bind Full Length Pdx-1

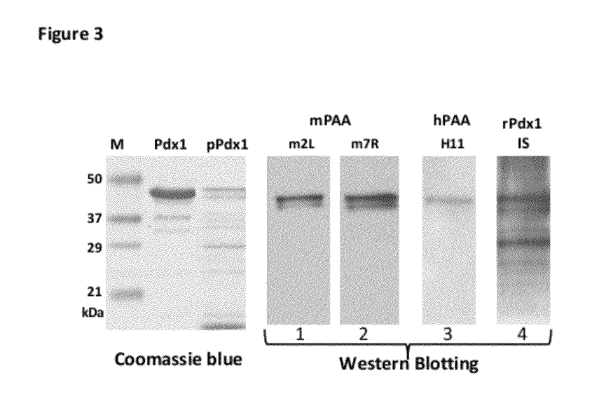

Western blot analysis showed that ELISA-positive NOD mouse sera recognized primarily the full-length Pdx1. These sera were examined as to whether they might recognize a common epitope. Full length recombinant Pdx1 (rPdx1) protein was partially digested with trypsin at 37 C. As shown in FIG. 3, 10 min digestion of Pdx1 generated multiple fragments that were

stained by Coomassie blue (left panel).

The trypsinized protein was also separated by SDS-PAGE, transferred to a nitrocellulose membrane and blotted with mouse sera and polyclonal immune serum against Pdx1 protein.

Interestingly, two NOD mouse samples (m2L and m7R) recognized the slowest migrating tryptic fragment of Pdx1 as well as full-length Pdx1. Without wishing to be bound by theory, this suggests that a specific site of Pdx1 protein is bound by Pdx1 autoantibodies.

In addition, 37 serum samples from patients (n=25) were also screened with well-established T1D or anti-insulin autoantibody (IAA)-positive individuals (n=12) for the presence of anti-Pdx1 antibodies by western blotting using purified full-length rat Pdx1 protein. Six serum samples were immunoreactive with Pdx1 protein: two samples (415) were from patients with long-standing T1D and four samples ( 4/12) from individuals with a positive test for IAA. To localize the immunodominant epitopes, partially trypsindigested Pdx1 protein was used as antigen. As shown in FIG. 3, human serum (H11) from a T1D patient recognized only full-length Pdx1, whereas rabbit anti-Pdx1 polyclonal immune serum was immunoreactive with nearly all of the fragments (FIG. 3, IS). Thus, the major autoepitope(s) recognized by NOD and human T1D sera are likely to be located near the C-terminus of Pdx1, as 19 out of 29 of the trypsin-cleavage sites (arginine or lysine) are located within amino acids 160-283.

### Example 4

### Pdx-1 Autoantibodies Bind Pdx-1 Amino Acids 200-283

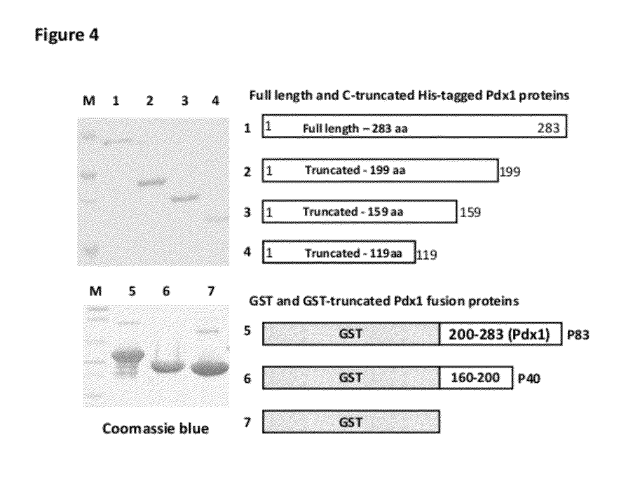

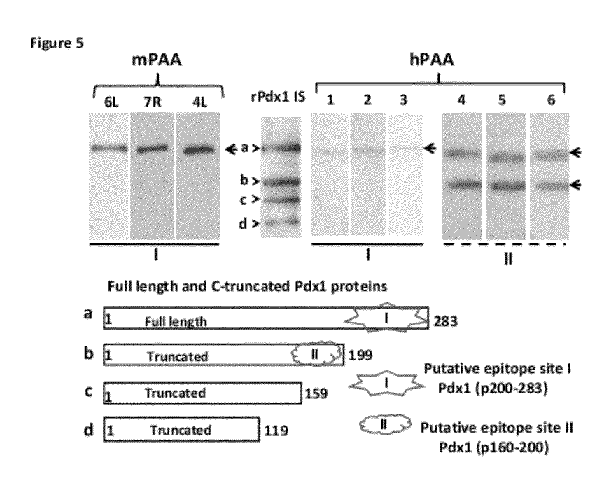

To map the B-cell autoepitopes, histidine-tagged C-terminal truncated Pdx1 proteins consisting of amino acids 1-119, 1-159, and 1-199 (FIG. 4, upper panel) were constructed, expressed, and purified. These truncated proteins were mixed with full-length rPdx1 for western blot analysis using three NOD-PAA+ mouse serum samples (FIG. 5, left panel) and six human PAA+ serum samples (FIG. 5, right panel). NOD mouse sera recognized primarily full-length rPdx1 protein (a), with weaker binding (7R) to the truncated Pdx1 protein (b, 1-199), but not the two shorter truncated Pdx1 proteins c (1-159) and d (1-119). In contrast, the rabbit polyclonal anti-Pdx1 immune serum (rPdx1 IS) recognized all four proteins. Serum from a negative NOD mouse did not react with any of the proteins, suggesting that the PAAs in mouse sera reacted with a major epitope (I) located within the C-terminal 84 amino acid residues and a minor epitope (II) located within Pdx1 (160-199). This result suggests that the binding site (epitope) against which autoantibodies are generated is within amino acids 200-283 of C-terminal Pdx1.

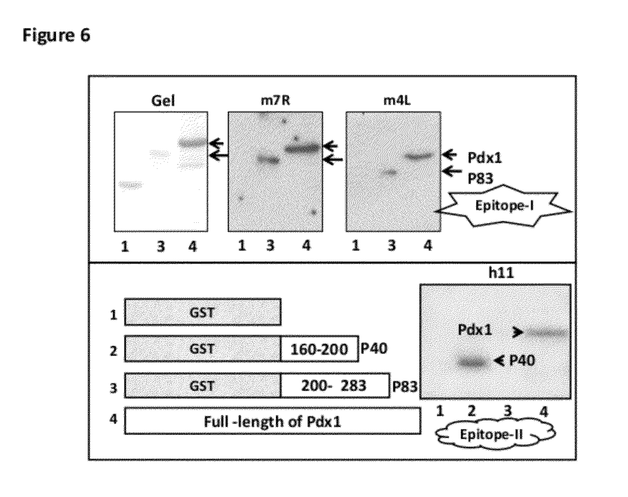

To exclude reactivity with the His-Tag or the possibility that amino acids 1-199 participate in forming a discontinuous epitope, a cDNA fragment encoding the C-terminal 84 amino acids (200-283) was inserted into the pGRx5.1 GST expression system. The GST expression plasmid contained a Factor X cutting site between GST and Pdx1 (200-283) sequence. The expression plasmid was confirmed by sequence analysis and the fusion protein was produced and purified according to the manufacturer's protocol. Using this protein without cleavage from GST, western blot analysis was performed, showing that autoimmune NOD sera bind strongly to the GST-Pdx1/200-283 or p83 protein but not to GST (FIG. 6, upper panel). These results indicate that the major autoepitope is located on the C-terminal portion of Pdx1 (amino acids 200-283) and immunoreactivity is not due to secondary structure.

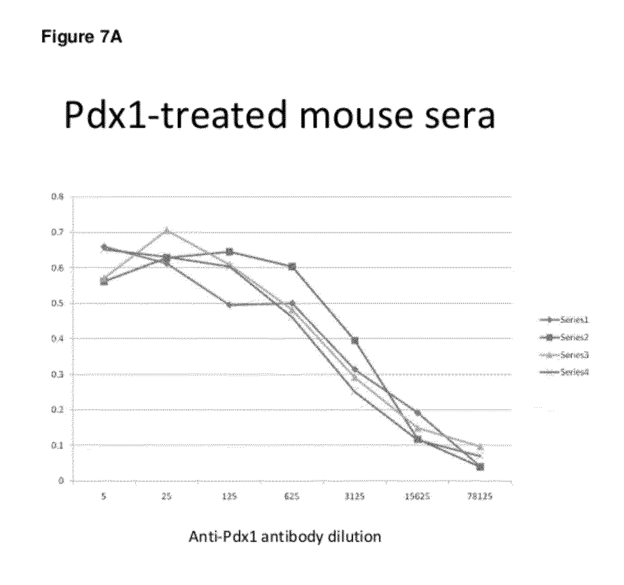

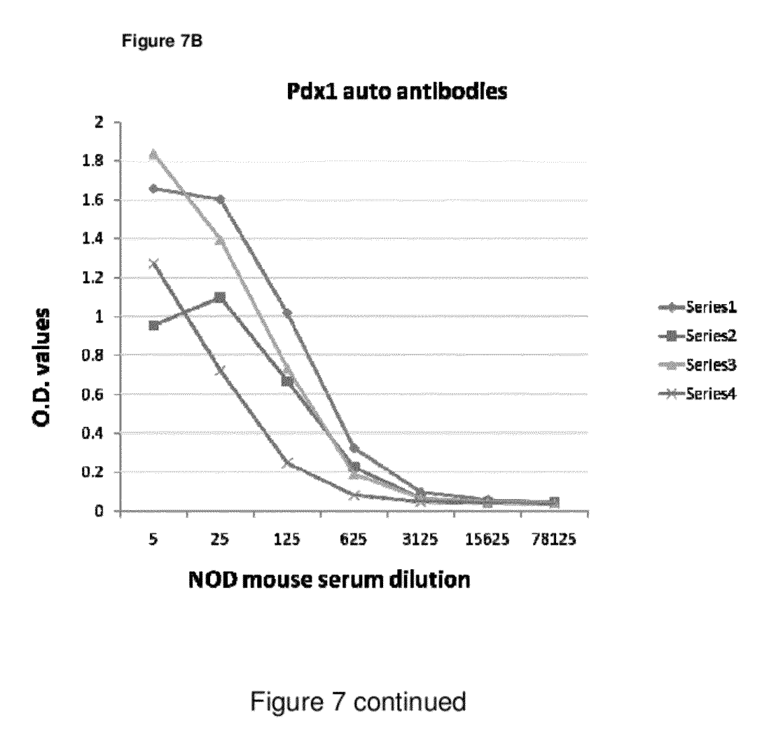

To determine whether PAAs from human sera recognize the same epitope bound by NOD mouse PAAs, a mixture of Pdx1 and its truncated forms was used for western blot analysis using PAA+ human sera of T1D patients and IAA-positive individuals. FIG. 5 (top right panel) shows two patterns among the six serum samples tested. Similar to mouse autoimmune sera, the human sera in lanes 1, 2, and 3 recognized only full-length Pdx1, indicating that they bind to an epitope or epitopes within the C-terminal 84 amino acids. Interestingly, human sera in lanes 4, 5, and 6 reacted equally with the top two bands, consistent with the existence of another epitope (II) located within amino acids 160-199. FIG. 5 (lower panel) illustrates the two putative Pdx1 autoepitopes recognized by human sera. To confirm the locations of these epitopes, cDNA encoding Pdx1 amino acids 160-199 (as p4-0) was inserted into the pGRx5.1 GST expression system. Using this fusion protein, western blot analysis was performed, showing that human sera (FIG. 5, lanes 4, 5, and 6) bind strongly to the GST-p40 protein and full-length Pdx1, but not to GST-p83 or GST alone, indicating that autoepitope II indeed is located within amino acids 160-199 of Pdx1 (FIG. 6, lower panel). FIGS. 7A and 7B are graphs showing ELISA results obtained following autoantibody dilution. These graphs show the Pdx-1 autoantibody is present in mouse sera at high titer and is highly specific for the Pdx-1 polypeptide.

### Example 5

### Pdx-1 is a Predictive Marker for Type 1 Diabetes

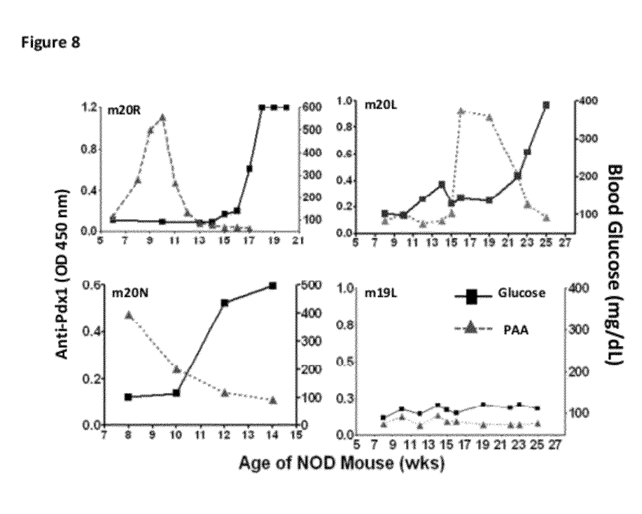

To determine whether the PAAs can predict disease onset, the relationship between PAAs and hyperglycemia was explored (FIG. 8). Five-week-old female NOD mice (n=20) were studied longitudinally and blood samples were taken biweekly until the onset of diabetes. Blood glucose levels were monitored weekly beginning at 10 weeks of age. PAAs were first detected at 5-15 weeks of age, and their levels gradually increased, peaked, and then declined over the next 8-12 weeks. PAAs often decreased to lower positive levels or disappeared completely after the onset of diabetes and the peak levels often preceded disease onset. In general, there was an inverse correlation over time between PAA levels and blood glucose levels in individual mouse. However, some mice maintained high levels of the PAAs after the onset of diabetes. FIG. 8 illustrates the relationship between levels of PAAs and blood glucose in four mice. In three mice, PAAs peaked before the onset of diabetes. In the fourth mouse (m19L), low levels of PAAs were seen consistently without an apparent peak; this mouse remained normoglycemic at 25 weeks. These results suggest that PAAs predict the onset of type 1 diabetes in NOD mice. Pdx1 autoantibodies appeared much earlier than the onset of diabetes indicating that Pdx1 autoantibodies in blood serum can serve as a diagnostic marker for prediction of type 1 diabetes.

### Example 6

### Pdx-1 Administration Treats Diabetes

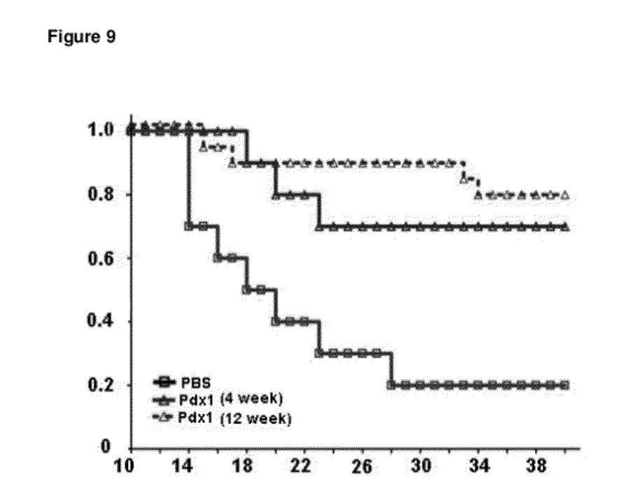

To determine if Pdx-1 administration could treat or prevent diabetes, Pdx-1 was administered daily to NOD mice at the age of ten weeks at 100 μg/day for 4 weeks or 12 weeks by intraperitoneal injection. Surprisingly, daily injections of Pdx-1 at this dosage and for this time period prevented diabetes. As shown in FIG. 9, both intraperitoneal Pdx1 administrations (IP for 4 week or 12 week) could delay the onset of diabetes, compared with NOD mice treated with PBS. Moreover, there existed no obvious differences of therapeutic effect between 4 week-treated group and 12 week-treated group.

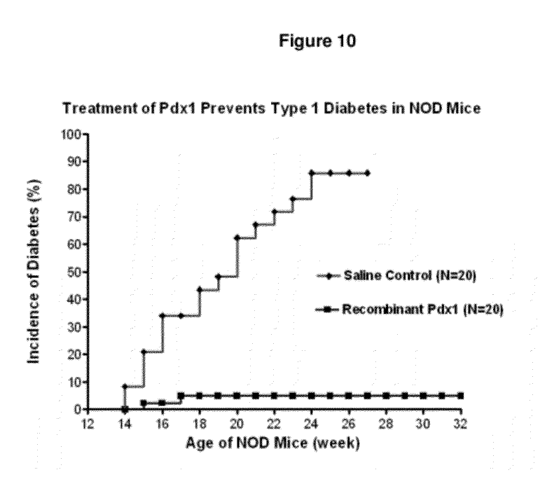

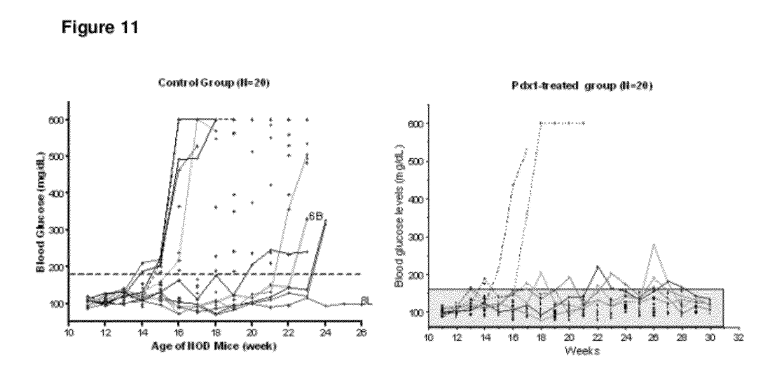

FIGS. 10 and 11 show that Pdx-1 administration reduced NOD mice blood glucose levels to within the normal range. Importantly, only 10% of NOD mice treated with Pdx-1 exhibited blood glucose levels indicative of diabetes (FIG. 10). In contrast, 95% of NOD mice exhibited blood glucose levels indicative of diabetes (FIG. 10). These results indicate that daily injections of Pdx-1 are useful to prevent or delay diabetes.

### Example 7

### Pdx-1 Treatment Reversed Diabetes in NOD Mice

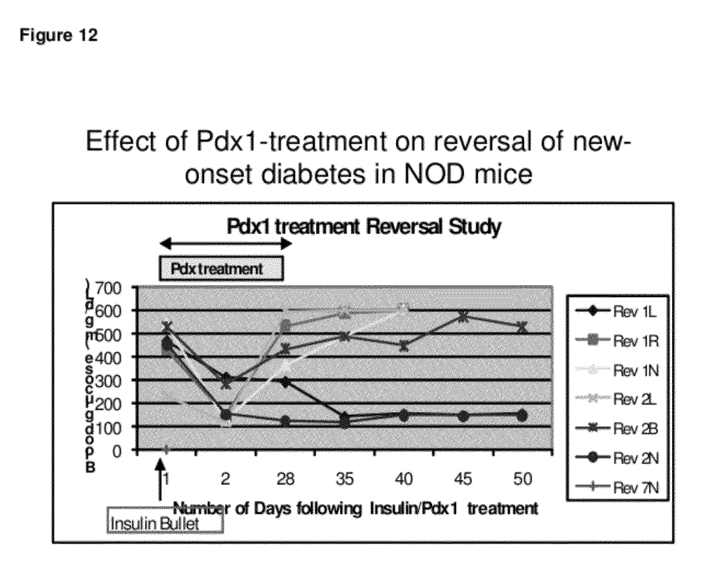

To determine whether Pdx-1 could reverse diabetes after diabetes onset, diabetic female NOD mice were subcutaneously implanted with an insulin bullet. The effect of insulin administered by this method usually lasts for 3 weeks. The mice also received daily injections of Pdx1 for 4 weeks (Dose: 100 ug per day per mouse). Blood glucose was monitored regularly. Surprisingly, this treatment regimen effectively treated the majority of mice (FIG. 12). Specifically, daily Pdx-1 administration completely reversed diabetes in 29% of diabetic mice (2 out of 7) as indicated by normoglycemia, and another 30% of diabetic mice showed an improvement in diabetes, as indicated by improved body weight and well-being. NOD mice with untreated diabetes typically show dramatic weight loss. In contrast, Pdx-1-treated NOD mice had stable body weights following treatment. In addition, Pdx-1 treated NOD mice were able to respond to a bolus dose of gluocose, whereas untreated NOD mice lacked this ability. In sum, at least 60% of NOD mice treated with Pdx-1 showed reversal or improvement in diabetes as judged by body weight and response to glucose challenge.

### Example 8

### Pdx-1 Homeobox is not Required for Diabetes Treatment

Pdx-1 is a transcription factor encoded by a Hox-like homeodomain gene. In humans and other animal species, the embryonic development of the pancreas requires PDX-1, as demonstrated by the identification of an individual with pancreatic agenesis resulting from a mutation that impaired the transcription of a functionally active PDX-1 protein. In adult subjects, PDX-1 is essential for normal pancreatic islet function as suggested by its regulatory action on the expression of a number of pancreatic genes, including insulin, somatostatin, islet amyloid polypeptide, the glucose transporter type 2 and glucokinase. To determine whether Pdx-1 transcriptional regulatory activity is required for the observed effect on diabetes, a Pdx-1 polypeptide having a mutation that blocked DNA binding was administered to NOD mice.

10-week-old female NOD mice were purchased from Jackson labs. Their body weight and blood glucose levels were recorded. The mice were divided into 3 groups: one group received recombinant rat Pdx1 protein, one group received mutant rat Pdx1 (mut-Pdx1) having a deletion of 16 amino acids within its homeodomain (Koya et al., Diabetes 57:757-769, 2008), and one group received saline control. Each group contained 10 mice. The mice received daily intraperitoneal injections of either 100 μg Pdx1, Mut-Pdx1 protein or saline for four weeks and their blood glucose levels were monitored weekly.

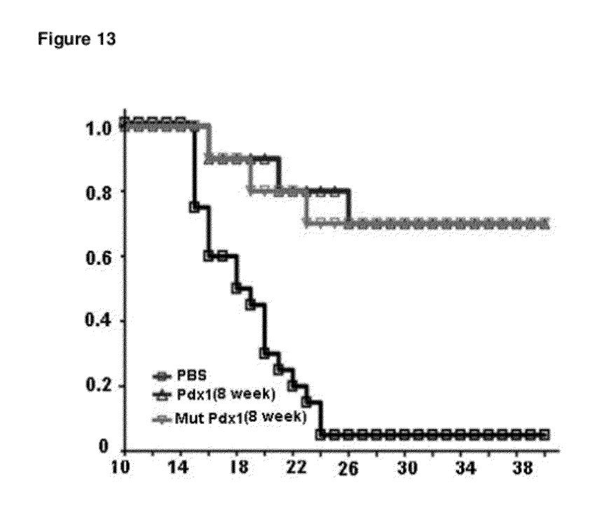

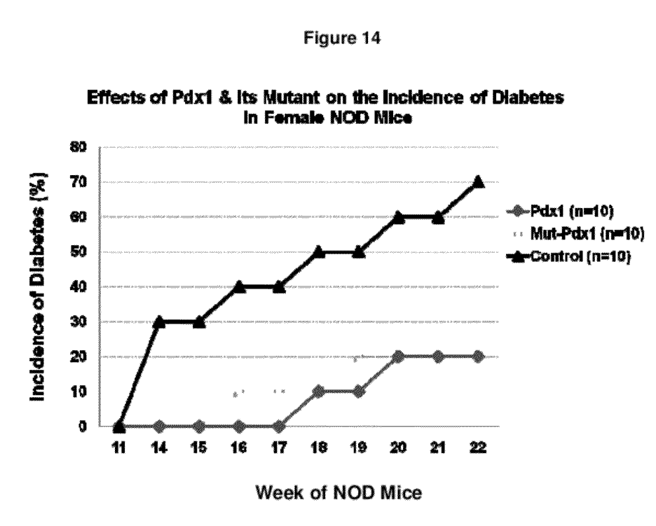

As shown in FIG. 13, Mut Pdx1 protein, which showed no biological function but intact immunologically active component, was administrated as a therapeutic protein into NOD mice. The results showed that both intraperitoneal Pdx1 and Mut Pdx1 administration (IP for 8 weeks) could delay the onset of diabetes, compared with NOD mice treated with PBS. Both Pdx1 and Pdx1 mutant proteins showed significant therapeutic effects (FIG. 14). In fact, administration of the Pdx1 or Pdx1 mutant protein prevented or delayed diabetes onset in female NOD mice relative to the saline control group. These results were surprising because the Pdx1 homeobox is required for Pdx1 biological function (i.e., transcriptional regulation). Although Pdx1-treated mice showed slightly better results than mutant Pdx1 group, these results indicate that Pdx1 transcriptional regulatory activity is not required for efficacy. Rather, these results suggest that Pdx1 is modulating the autoimmune response in NOD mice.

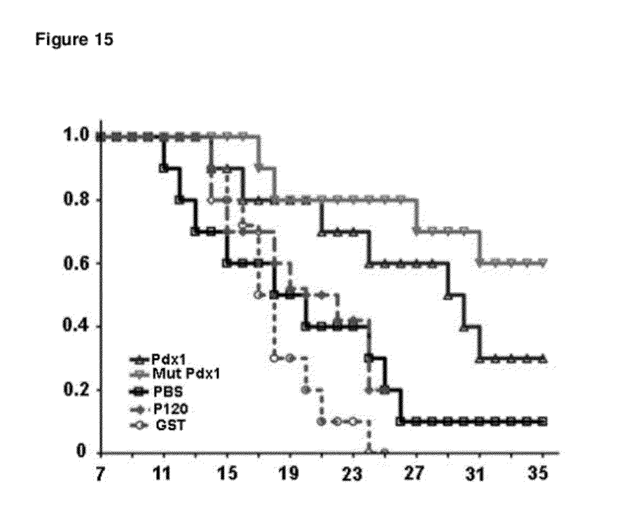

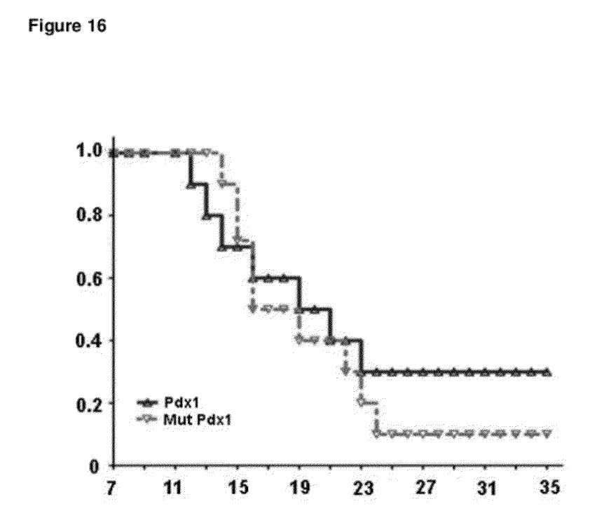

As shown in FIG. 15, NOD mice at the age of seven week were injected subcutaneously with Pdx1 protein or Mut Pdx1 protein daily for 4 weeks, and subsequently twice a week for 4 weeks. Similarly processed GST and P120 were chosen as control proteins. The result showed that subcutaneous administration of Pdx1 protein or Mut Pdx1 protein could delay the onset of diabetes in NOD mice over the 35-week study period, and the protection against diabetes was more obvious by immunizing with Mut Pdx 1 protein. However, prevention of diabetes was not achieved in the GST, P120 or PBS-treated groups. All mice in the control groups gradually lost body weight and died spontaneously due to high glucose levels. As shown in FIG. 16, oral administrations of Pdx1 did not prevent diabetes. To sum up, the above data suggested that Pdx1 treatment could delay the new-onset diabetes in spontaneous diabetic NOD mice and this prevention was Pdx1 antigen-specific.

### Example 9

### Pdx-1 has an Immunomodulatory Effect

Six-week-old NOD-SCID mice (n=5/group) were divided into two treatment groups, each of which received 1×10⁶ splenocytes through the tail vein injection. In the first group, splenocytes were isolated from Pdx1-treated NOD mice via splenectomy. The Pdx-1 treated mice were 25-weeks-old and were normoglycemic following daily intraperitoneal injection with Pdx1 protein beginning at 12 weeks of age until 25 weeks of age. This treatment was carried out 10 weeks prior to the surgery. Splenocytes for the control group were obtained from NOD-SCID mice that showed recent onset of diabetes.

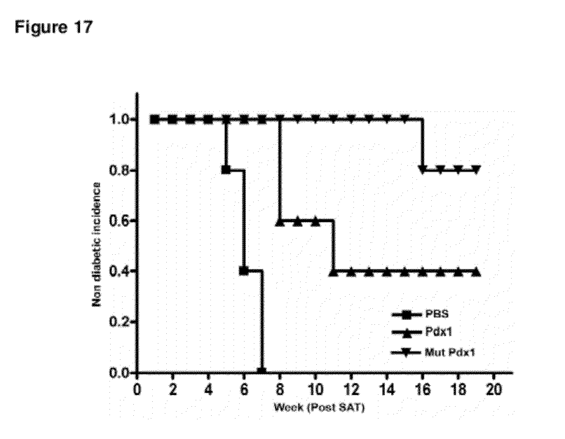

Ameliorating autoimmunity through immunoregulation was assessed by adoptive transfer. To confirm therapeutic effect of Pdx1 protein immunizations, 5-week-old NOD-SCID mice were injected with splenocytes from immunized NOD mice treated with Pdx1 protein (Mut Pdx1 protein or PBS) for 4 week. Non-diabetic incidence analysis revealed a significant delay in the onset of diabetes among the mice that received infusions of splenocytes from Pdx1 or Mut Pdx1-immunized donors (FIG. 17). However, NOD-SCID mice injected with splenocytes from PBS-treated NOD mice showed no significant therapeutic effect, and most of mice become diabetic within 3 weeks of cell transfer. This result suggested the activation of immunoregulatory cells in NOD mice treated with Pdx1 protein, and protection from diabetes could be adoptively transferred to NOD-SCID recipients. Thus, immunoregulation through lymphocytes may play an important role in preventing diabetes in NOD mice treated with Pdx1 protein.

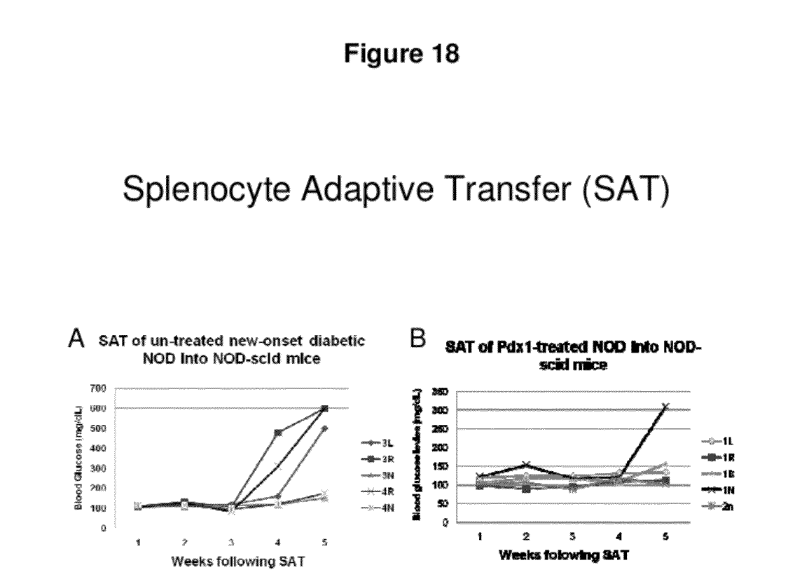

Blood glucose levels were monitored weekly via a glutometer. Random normal blood glucose levels for NOD-scid mice range from 98 to 120 mg/dL. Abnormal blood glucose levels are defined twice measurement s>150 mg/dL after first day abnormal reading. Random glucose levels above 250 mg/dL are defined onset of clinical diabetes. By 4 weeks after splenocyte adaptive transfer (SAT), 40% of control group mice became diabetic (FIGS. 18A and 18B). Surprisingly, no mice from Pdx1-treated SAT were diabetic (FIGS. 18A and 18B). By five weeks, 60% of control mice were overtly diabetic, whereas only 20% of Pdx1-treated SAT mice were diabetic. These results indicated that Pdx1 treatment has an immunodulatory effect on the diabetogenic T-lymphocytes, possibly by increasing T-regulatory lymphocytes.

### Example 10

### Pdx1 Amino Acids 200-283 Stimulate Lymphocyte Proliferation

To determine whether Pdx1 can stimulate antigen-specific T-cell proliferation, freshly isolated splenocytes from new-onset diabetic NOD mice were stimulated with various forms of Pdx1 protein including full-length Pdx1 and different truncated forms. Anti-CD3 antibodies were used as a positive control for T-cell proliferation, and either untreated or GST-treated (unrelated protein) T cells were used as a negative control. ³H thymidine incorporation was used to measure cell proliferation. Splenocytes isolated from NOD diabetic spleens were plated in 96-well plate with 1 million cells/well. Various antigens (Pdx1 full length or different truncated forms) were added into the wells. Each condition were tested in triplicate. Cells were cultured for 48 hrs before 1 μCi/well of ³H-thymidine was added to each well.

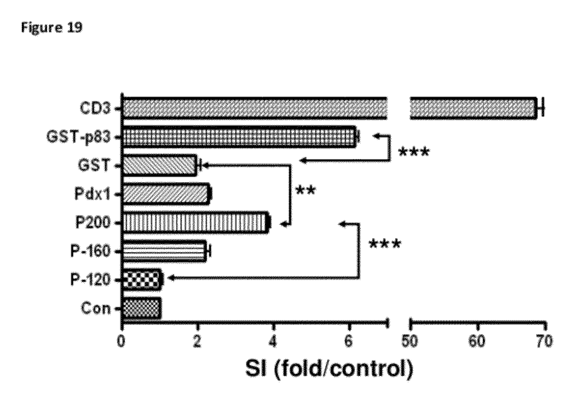

FIG. 19 shows that full-length as well as truncated forms of Pdx1 can stimulate T-lymphocyte proliferation. However, the C-terminus of Pdx1 (200-283, or p83) had the most potent T-cell stimulatory effect (almost sevenfold increase over control GST, P<0.00 1 by Student's t-test), suggesting that the major T-cell epitope(s) are located in close proximity to the major B-cell autoepitope. Interestingly, a significant T-cell stimulatory effect also was observed in the wells containing p200 (four times over control, P<0.001 by Student's t-test), supporting the possible presence of minor autoepitopes within the region of p160-200.

These results indicate that Pdx1 is an autoantigen in NOD mice and plays a pathogenic role in causing type I diabetes. Pdx1 amino acids 200-283 specifically bind autoantibodies in mouse sera and amino acids 150-200 in human sera. Treatment of NOD mice with autoantigen(s) of Pdx1 prevented or delayed the onset of T1D. Therefore, various forms of Pdx1 can be used as therapeutic reagent for prevention or treatment of T1D.

### Example 11

### Pdx1 Protein Treatment Diminishes Insulitis

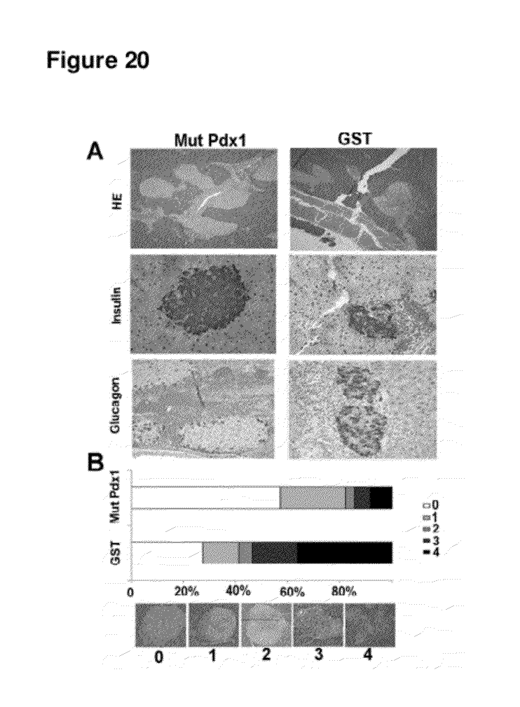

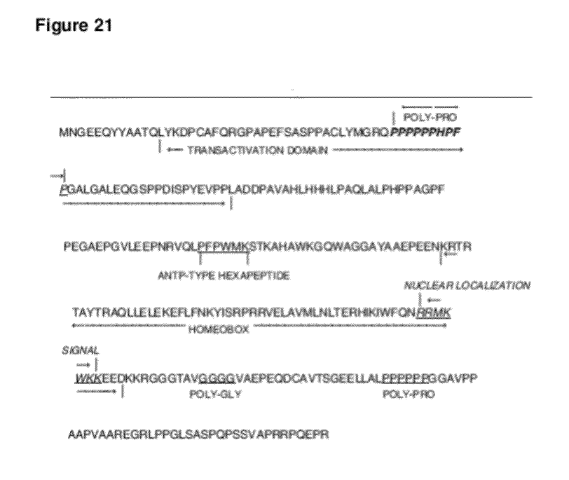

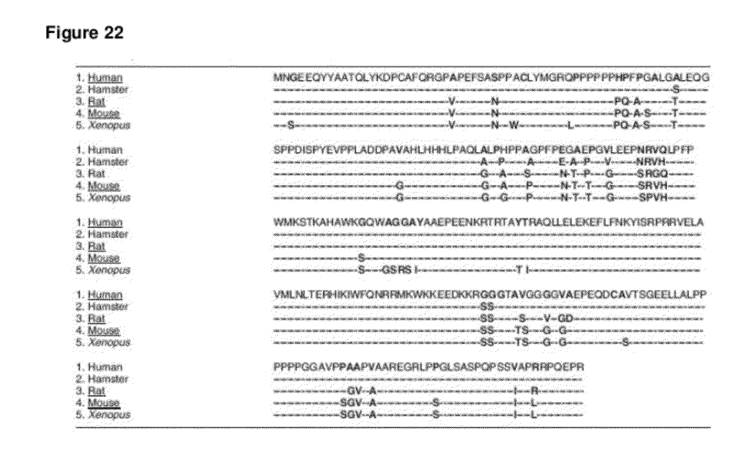

In Mut Pdx1 protein treated group, eight weeks after the first GST injection, histological examination of pancreas revealed islets heavily infiltrated by leukocytes (FIG. 20A). The few islets with remaining insulin containing β-cells were infiltrated by abundant leukocytes, and most of these islet cells contained glucagon-positive cells. In contrast, in Mut Pdx1 protein treated mice, the extent of lymphocyte infiltration of the islets was reduced, and immunohistochemistry of pancreas revealed abundant insulin containing β-cells. Insulitis severity scores (FIG. 20B) in NOD mice treated with Mut Pdx1 (n=15) were much better than that in GST treated group.

### Example 12

### Luminescence Immunoprecipitation System (LIPS) Assay Provides a Sensitive, Specific, and Non-Radioactive Assay for Detection of PAA in Human Sera

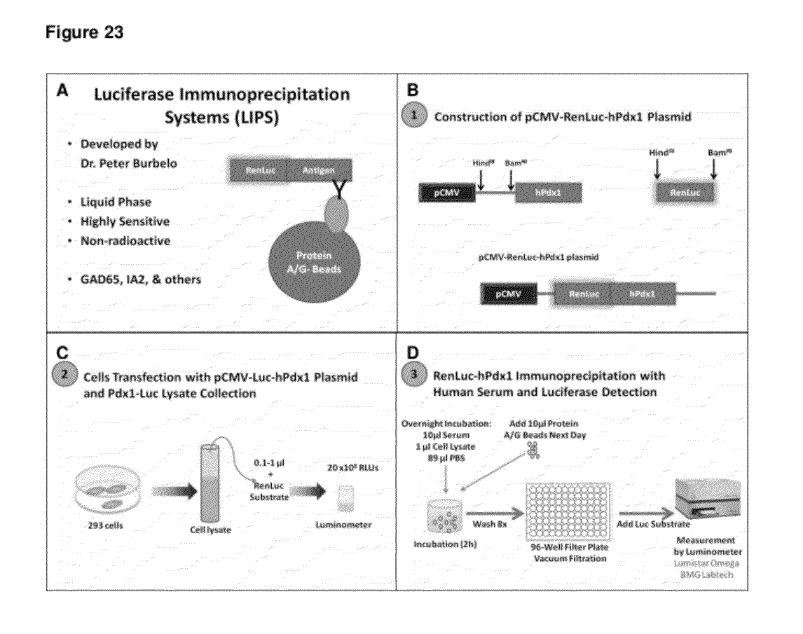

A liquid-phase luminescence immunoprecipitation system (LIPS) assay was developed for detection of PAA in human sera using a Pdx1-luciferase fusion protein (FIG. 23A). The results herein demonstrate that LIPS provides a sensitive, specific, and non-radioactive assay for detection of PAA in human sera.

Several plasmids were constructed that are useful for detecting PAA from human sera by fusing the renilla luciferase gene to human or mouse Pdx1 in both mammalian (pCMV) and bacterial (T7) expression systems (FIG. 23B). Renilla luciferase only expression plasmids were constructed by introducing a stop codon directly following the renilla luciferase coding sequence. These plasmids were used in transfections to generate respective protein containing lysates from both mammalian cells and bacteria as described in the methods (FIG. 23C). Transfection conditions were optimized to yield lysate with high RLU values when activated (up to 10⁷ RLU/0.1 μl). E. coli produced renilla luciferase-mPdx1 fusion protein lysate was tested by performing a standard LIPS assay using serial dilutions of polyclonal rabbit serum containing Pdx1 antibodies (FIG. 23D). A dose-response curve demonstrated that renilla luciferase-mPdx1 fusion protein lysate was working properly and able to detect Pdx1 antibodies in serum.

### Example 13

### Luciferase Immunoprecipitation Systems (LIPS) Assay is as Sensitive as Radioimmunoprecipitation (RIA) for Autoantibody Detection

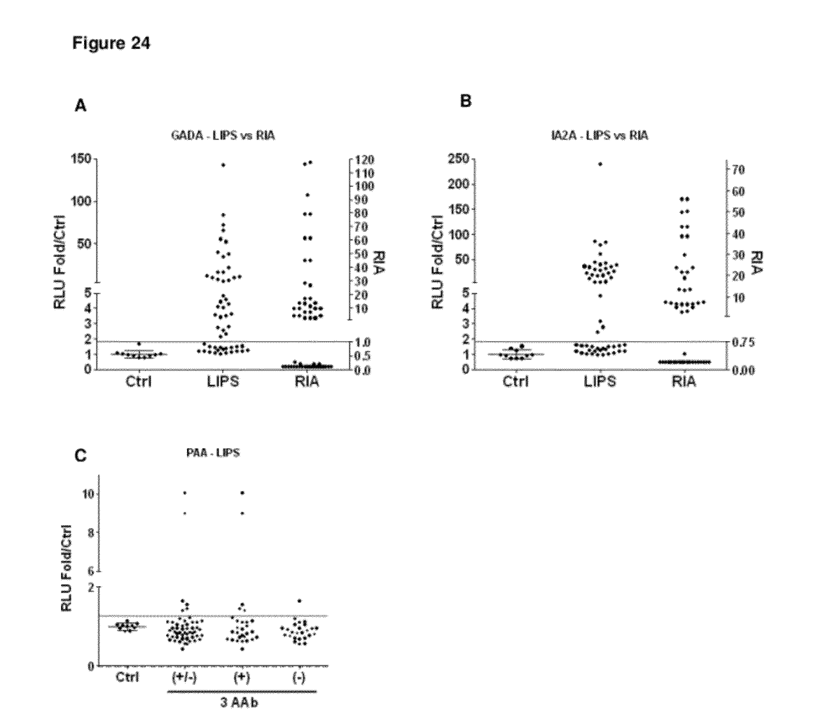

To detect GADA and IA-2A, LIPS assay was used to blindly measure 54 clinical sera (10) that had been determined as positive (or negative) for ICA, GADA, and IA-2A according to clinical data (FIGS. 24A and 24B). Human sera (from 10 normal, healthy donors) were used as controls to set a 3 standard deviation above mean cutoff to assess positive sera from the 54 clinical samples. Of 29 serum samples determined as clinically positive for GADA, 28 serum samples (97%) were identified by LIPS as positive, as well as 6 additional samples that were determined as clinically negative. For IA-2A, all 29 clinically positive sera (100%) were also identified as positive by LIPS, as well as 1 additional sample that was determined as clinically negative. The sensitivity for individual samples varied greatly between the clinical data and LIPS assay without significant correlation. For example, a particular sample may yield a high positive clinical signal by RIA but a low-moderate signal by LIPS assay, or vice versa.

If the clinical RIA is held as the standard, LIPS assay has nearly identical sensitivity but a reduced specificity because several false positive sera were identified. However, an alternative interpretation of the data is that the LIPS assay is more sensitive than RIA and that these “false positive” samples were actually true positive samples that are undetectable by RIA. Lending support to the increased sensitivity of LIPS over RIA is the fact that the “clinically negative” patients presented diabetic symptoms, and, thus, were tested for the presence of T1D related autoantibodies. Therefore, the patients with “clinically negative” sera may have T1D autoantibodies but at low titer and undectectable by RIA. Using the LIPS assay as the standard, clinical RIA has 97% sensitivity and 76% specificity for GADA detection and 100% sensitivity and 96% specificity for IA-2A detection. Because the LIPS assay is more sensitive and safer than RIA it may be useful to introduce in the clinical setting in the future. Altogether, the data suggests that the LIPS assay is at least capable of performing with similar sensitivity of detection to clinical RIA.

### Example 14

### Luciferase Immunoprecipitation Systems (LIPS) Assay is as Sensitive as Radioimmunoprecipitation (RIA) for Autoantibody Detection

The LIPS assay was used to measure the 54 clinical sera and 10 normal human sera (10 μl) for PAA using E. coli produced renilla luciferase-mPdx1 fusion protein lysate. Using a 3 standard deviation cutoff above normal human controls, 24 (44%) of these sera were found as positive for PAA (FIG. 24C). Because the 54 clinical sera were either determined as positive for all three T1D markers (ICA, GAD65, and IA2) or negative for all three T1D markers, the correlation between these three markers with PAA we next assessed (FIG. 24C). Of the serum samples positive for PAA, 34% of the sera were positive for the three T1D markers, and 56% of the sera were negative for the three T1D markers. Thus, no obvious correlation between the presence of PAA and other clinically relevant autoantibodies was observed. Using the mammalian produced luciferase-hPdx1 fusion protein lysate, 7 (13%) of the sera were found positive for PAA. In this case, 21% of sera positive for the three T1D markers were also positive for PAA, and 3% of sera negative for the three T1D markers were positive for PAA. The fact that 6 out of 7 (86%) PAA positive sera were also positive for the 3 other T1D markers suggested some correlation between the presence of PAA with the other T1D related autoantibodies. However, a great difference in number of positive PAA sera were identified when comparing LIPS assays using E. coli and mammalian produced fusion protein lysate. Further studies could be performed to confirm a correlation between the presence of PAA and other T1D related autoantibodies.

An interesting observation is seen when comparing the detection ability of GADA and IA-2A versus PAA. GADA and IA-2A have been observed to often be high titer, high affinity autoantibodies (FIGS. 24A and 24B). GADA and IA-2A was detected at 150-250 fold, respectively, above normal human controls using only 1 μl of patient serum. GADA and IA-2A have also been proposed to be generated as a result of an epitope spreading following primary immune attack and subsequent presentation of new autoepitopes to the immune system. Because GAD65 and IA2 are expressed in several tissues, these antigens can continue to stimulate the production of autoantibodies and persist long term. In contrast, when using 10 ul of patient serum, PAA was only detectable up to 15 fold above normal human controls (FIG. 24C). Because it is common for autoantibodies to be low titer and low affinity antibodies, this could be expected. As opposed to GAD65 and IA2, Pdx1 expression is restricted to few tissues types and is a key transcription factor in pancreatic beta cells.

Without intending to be bound by theory, Pdx1 is an early autoantigen in respect to T1D development. Insulin is also considered to be an early autoantigen in T1D and it has been proposed to be the primary antigen related to T1D. This is based on its early detection in T1D and the fact that it is uniquely expressed and secreted by beta cells. However, there are several issues regarding insulin autoantibody (IAA) detection. First, many traditional assays are not able to detect IAA possibly due to denaturing conditions. Second, dramatic inconsistencies following IAA detection have been reported. And third, once a patient has been placed on insulin replacement therapy, IAA can no longer be evaluated due to the immune response to the exogenous insulin delivery.

There is evidence that Pdx1 is the primary autoantigen related to T1D. PAA in NOD mice were detected by ELISA, western blotting, and radioimmunoprecipitation. PAA titers of NOD mice during the development of diabetes peaked prior to the onset of hyperglycemia and then dropped to undetectable levels after several weeks. Without intending to be bound to theory, this effect is likely due to lack of Pdx1 antigen stimulation following destruction of beta cells. Because PAA is a primary marker for T1D, sensitive detection of PAA is useful for diagnosing T1D.

### Example 14

### Control and Competition Experiments Confirm the Sensitivity of LIPS for PAA Detection

Two separate methods were used to confirm the specificity of LIPS for PAA detection in human sera. One method demonstrated that renilla luciferase-only lysate (without Pdx1 antigen) could not be used to detect PAA. One negative and three individual high-signal positive sera that previously tested positive for PAA were selected. These sera were then assayed by LIPS using either renilla luciferase-only or renilla luciferase-mPdx1 fusion protein lysate from E. coli as well as renilla luciferase-only or renilla luciferase-hPdx1 fusion protein lysate from mammalian cells. Using the renilla luciferase-only lysates, PAA could not be detected, while renilla luciferase-Pdx1 fusion protein lysates were able to detect PAA in all three positive sera, demonstrating specificity for Pdx1 antigen.

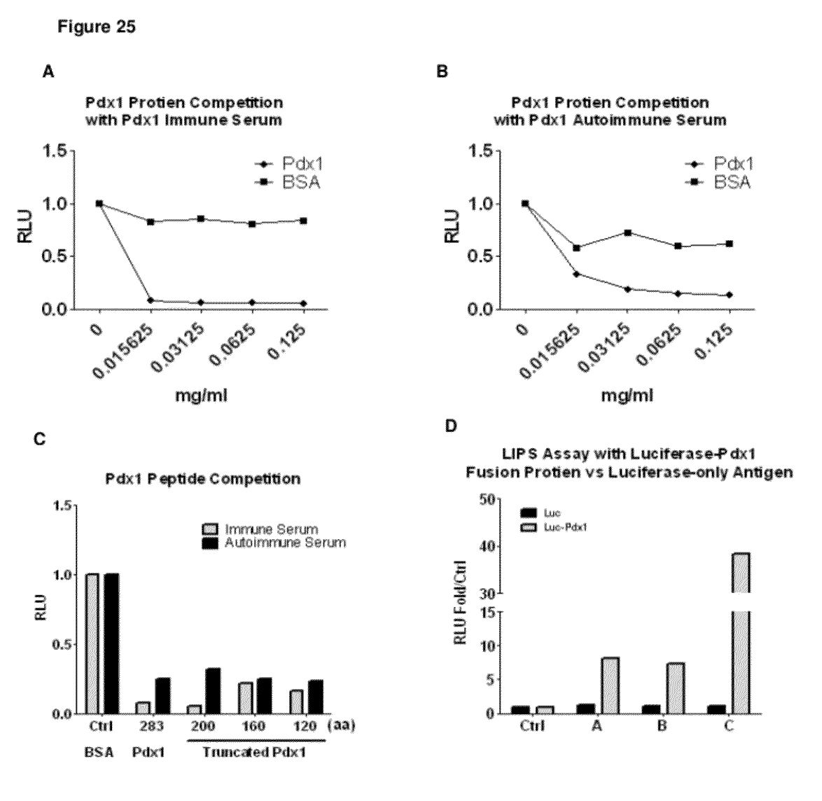

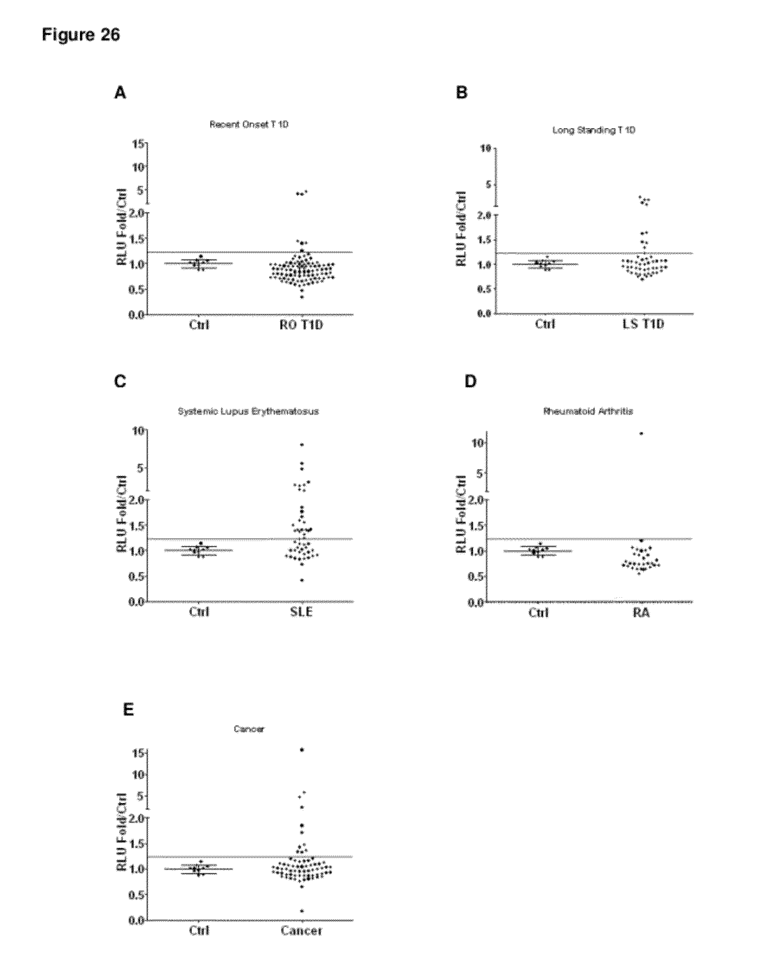

A second method to confirm Pdx1 specificity in the LIPS assay demonstrated that unlabeled (no luciferase) recombinant rat (r)Pdx1 purified protein (15), but not BSA (non-specific protein), could block PAA detection. This blocking system was demonstrated by performing a standard LIPS assay with E. coli produced renilla luciferase-mPdx1 and polyclonal rabbit serum containing Pdx1 antibodies in addition to indicated concentrations of purified rPdx1 or BSA (FIG. 25A). Excess purified rPdx1 protein competed with renilla luciferase-mPdx1 fusion protein for Pdx1 antibodies while BSA did not. A standard LIPS assay was performed with E. coli produced renilla luciferase-mPdx1 and sera from a high signal positive patient with long standing T1D in addition to indicated concentrations of purified rPdx1 or BSA (FIG. 24B). The results demonstrate that purified rPdx1 protein was able to compete with renilla luciferase-mPdx1 fusion protein for PAA. In this case BSA reduced signal but was unable to block detection, even at high concentration (0.125 mg/ml).

To investigate further the location of the Pdx1 autoepitope, standard LIPS assay was performed using E. coli produced renilla luciferase-mPdx1 and either Pdx1 Ab+ rabbit sera or sera from a high-signal positive patient with long standing (LS) T1D. For competition studies, 10 μg/μl full length purified rPdx1 (283), purified rPdx1 with various C-terminal truncations (200, 160, or 120), or BSA were added. A control LIPS assay was also performed using E. coli produced renilla luciferase-mPdx1 and polyclonal rabbit serum containing Pdx11 antibodies. Full length Pdx1 and all truncated constructs were expected to block detection of Pdx1 polyclonal antibodies and only the truncated constructs containing an autoepitope were expected to block detection of PAA. However, all truncated rPdx1 proteins unexpectedly blocked both Pdx1 polyclonal antibodies and PAA (FIG. 24C,). Without intending to be bound to theory, from this data (for this particular patient), it is likely that either the autoepitope exists within the first 120 amino acids of the Pdx1 protein or multiple autoepitopes exist.

### Example 15

### Pdx1 Autoantibodies was Detected in Human Sera of Patients with Recent-Onset T1D, Long-Standing T1D, Systemic Lupus Erythematosus, Rheumatoid Arthritis, and Pancreatic Cancer

To determine if the presence of PAA is unique to T1D, standard LIPS assay was performed using mammalian produced renilla luciferase-hPdx1 fusion protein lysate to detect PAA in human sera from recent onset (RO) T1D patients (FIG. 25A), LS T1D patients (FIG. 25B), patients with systemic lupus erythematosus (SLE) (FIG. 25C), patients with rheumatoid arthritis (RO) (FIG. 25D), and patients with various forms of cancer (FIG. 25E). Using a cutoff of three standard deviations above the mean of normal human control sera, positive PAA sera were detected as follows: 7% from RO T1D patients (n=100), 20% from LS T1D patients (n=50), 48% from SLE patients (n=48), 3% from RA patients (n=30), and 16% from cancer patients (n=70). Sera that produced high positive signal for detection were assayed several times whenever possible (based on serum volume) and consistent results were produced. PAA serum with the highest detectable signal came from a patient with pancreatic cancer (15 fold over control). Without intending to be bound to theory, injury from pancreatic cancer could have caused leaking of Pdx1 protein and subsequent presentation to the immune system leading to production of PAA. Over production of Pdx1 from pancreatic cancer cells could also lead to high titer PAA production by B-cells. Thus, detection of PAA is useful for screening and diagnosing pancreatic cancer which remains one of the most elusive forms of cancer with a poor prognosis and 5 year survival rate less than 5%.

Results reported herein were obtained using the following methods and materials unless indicated otherwise.

### Serum Samples and Animals

Serum samples were collected from female NOD mice and congenic nondiabetic NOD-scid, BALB/c, and C57BL/6 mice (Jackson Laboratory, Bar Harbor, Me., USA). Mice were housed in an SPF facility at the University of Florida. Serum samples from female NOD mice were tested for PAAs at ages ranging from 5 to 25 weeks. Blood glucose was determined weekly starting at 10 weeks of age. Collection of serum samples from human subjects was approved by the Institutional Review Boards, and all animal experiments were approved by the Institutional Animal Care and Use Committee of the University of Florida.

### Preparation of Recombinant Pdx1, Mutant Pdx1, and Truncated Proteins

Complementary DNA fragments of rat or human Pdx1 were inserted into the expression vector pET28 (Invitrogen, Carlsbad, Calif., USA) and 6× histidine-tagged recombinant Pdx1, mutant Pdx1 lacking the protein transduction domain (Pdx1DPTD), and truncated Pdx1 proteins were produced, purified, and characterized. In brief, C-terminal Pdx1 truncations were generated by site-directed mutagenesis using a QuikChange Kit (Stratagene) by introducing a stop codon (TAG) mutation into rat Pdx1 cDNA at the amino-acid position 120, 160, or 200. The mutant cDNAs were verified by DNA sequence analysis. Three truncated Pdx1 fragments (1-119, 1-159, and 1-199) were expressed in the pET28 expression vector in Escherichia coli BL21(DE3) cells and purified by Ni-NTA affinity chromatography.

### Protein Expression and Purification of GST-P83 and GST-P40

Rat Pdx1, amino acids 200-283 (p83), and Pdx1, amino acids 160-199 (p40), were expressed in E. coli as fusion proteins to glutathione-S-transferase (GST). A fragment of the cDNA encoding the amino acids 200-283 or 160-199 of Pdx1 was amplified by PCR and ligated into the BamHI/XhoI sites of pGEX-5x-1 expression vector (Promega) for generating fusion proteins GST-p83 and GST-p40, respectively. A 6× histidine tag was attached to the N-terminus of GST-p40 to facilitate purification of this insoluble protein. The plasmids were transformed into E. coli BL21 (DE3) cells and the fusion proteins were produced. GST and GST-p83 fusion proteins were purified using a glutathione-Sepharose affinity purification kit (Pierce, Rockford, Ill., USA). Bound protein was eluted with 10 mM reduced glutathione in 50 mM Tris-HCl (pH 8.0) and dialyzed against PBS. GSTp40 was purified by Ni-NTA affinity chromatography under denaturing conditions. Protein concentration was determined and the purified proteins were snap-frozen and stored in aliquots at −80° C.

Pdx1 and its truncated forms were separated by 12% SDS-polyacrylamide gel electrophoresis (PAGE) and transferred onto nitrocellulose membranes (Bio-Rad). Blots were probed with mouse or human sera sample or with rabbit anti-Pdx1 antibodies (1:1000). The membrane was blocked with 5% nonfat dry milk (Bio-Rad) in Tris-buffered saline (TBS, pH 7.5; Bio-Rad), and then incubated with sera from mice (1:1500 dilution) or human T1D patients (1:200 dilution) overnight at 4° C. After washing five times with TBS containing 0.1% Tween-20, the membrane was incubated with horseradish peroxidase (HRP)-conjugated goat anti-mouse-IgG (1:4000, Abcam, Cambridge, Mass., USA) or anti-human IgG (1:2000, South Biotech) for 30 min at 22° C. Binding was detected by chemiluminescence (Amersham, Piscataway, N.J., USA).

### ELISA for Autoantibodies Against Pdx1

Autoantibodies against Pdx1 were quantified by ELISA. In brief, a microtiter plate (Nunc MaxiSorp, Fisher) was coated with 100 μμl of rPdx1 (10 μg/ml) overnight at 4° C. After washing the plate three times with PBS, the plate was incubated with 200 μl of 5% dry milk in PBS (blocking buffer) for 1 h. Next, mouse sera were added in duplicate at a 1:30 dilution or after serial dilutions in the blocking buffer for 1 h. The bound antibodies were incubated with HRP-conjugated goat anti-mouse IgG antibodies (1:4000 in blocking buffer, Abcam) or HRP-goat-anti-mouse IgG isotype-specific antibody kit (IgG1-, IgG2a-, IgG2b-, and IgG3-specific; Santa Cruz Biotechnology, Santa Cruz, Calif., USA) for 1 h at 22° C. After washing five times, the plate was incubated with 100 ul of substrate solution (BD OptEIA, BD Biosciences Pharmingen, San Diego, Calif., USA) and developed at 22° C. for 10 min. The reaction was terminated by adding 50 ul of 4 M H₂SO₄, and absorbance was determined at 450 nm. A positive result was defined as an ^{OD}450 nm 0.1 (the mean of BALB/c sera or istotype controls [symbol] 3 s.d.). Intra- and interassay variation (CV %) was determined with the same lot of ELISA plates and the same serum-positive and serum-negative samples.

### [³⁵S]-Labeling and Immunoprecipitation

Rat insulinoma INS-1 cells (clone 832/13, a gift of Christopher B Newgard, Duke University) were metabolically labeled overnight with [³⁵S] methionine plus [³⁵S]cysteine (GE Healthcare, Piscataway, N.J., USA). [³⁵S]-labeled INS-1 cell lysate containing rat Pdx1 protein (2000 volume, 10⁶ cell equivalents) were incubated with preformed complexes of immunoglobulin/protein A/G-Sepharose for 2 h at 4° C. Protein A/G-Sepharose was incubated with 10 μl of mouse sera or Pdx1-treated mouse serum (prepared in our lab) overnight. After centrifuging, 20 μl of 50% protein A/GSepharose beads (Pharmacia) were added followed by incubation at 22° C. for 60 min. Immune complexes were collected by centrifugation and washed three times. Proteins were freed by boiling in a sample buffer and analyzed on 12.5% SDS-polyacrylamide gels. The gels were stained and then fluorographed, followed by the exposure to X-ray film for 1 week. Similar immunoprecipitation (IP) was performed using unlabeled INS-1 cell lysates, and the immunoprecipitated Pdx1 protein from the cell lysates by the tested mouse sera was probed by western blotting using rabbit anti-Pdx1 polyclonal antibodies (1:2000 dilution), following separation by SDS-PAGE and transferring onto nitrocellulose membranes.

### T-Cell Proliferation Assay

New-onset diabetic female NOD mice were killed and splenocytes were harvested. The splenocytes (10⁶ cells/well in triplicate) were incubated for 72 h at 37° C. in a humidified atmosphere with 5% CO₂ in the absence or presence of various stimuli (see Results). Anti-CD3 antibody treatment was used as a positive control. T-cell proliferation was quantified by the incorporation of [³H]thymidine (Amer-sham, 1 mCi/well) for the last 24 h of incubation. The stimulation index (SI) was calculated as the ratio of the mean c.p.m. of antigen- or mitogen-treated cells divided by the mean c.p.m. of control cells cultured with medium alone.

### Non-Diabetic Incidence

Female NOD mice were purchased from Jackson Laboratory (Bar Harbor, Me.). NOD mice at the age of seven weeks or ten weeks were treated with (intraperitoneally or subcutaneously) Pdx1 protein (or Mut Pdx1 protein) for different weeks (n=10/each group). Littermate controls received commercial PBS diluent, P120 protein or GST protein (n=10/each group).

For oral administration of Pdx1 protein or Mut Pdx1 protein, a volume of 0.2 ml of 100 μg Pdx1 protein or Mut Pdx1 protein was administered with a syringe daily for 4 weeks, and subsequently twice a week for 4 weeks (n=10/each group). Blood glucose levels were monitored weekly, and mice with a blood glucose level>11.1 mmol/l (200 mg/dl) for 2 consecutive days were considered diabetic.

To investigate the underlying mechanism, immunizations with equivalent doses of Mut Pdx1 protein were performed (n=15), whereas controls received injections of equimolar amounts of GST (n=15). NOD mice at the age of seven week were injected subcutaneously with Pdx1 protein or Mut Pdx1 protein daily for 4 weeks, and subsequently twice a week for 4 weeks. Eight weeks after the first treatment, mice were killed, and histology of pancreas, cell flow and real-time PCR were performed to study the underlying mechanism.

### Adoptive T-Cell Transfer

NOD mice at the age of seven weeks were immunized with Pdx1 protein (or Mut Pdx1 protein) for 4 weeks. At 11 weeks of age, mice were killed and spleen cells were isolated. NOD-SCID female mice at the age of five were injected intraperitoneally with 1.5×10⁷ spleen cells from Pdx1 (or Mut-Pdx1) treated NOD mice. The development of diabetes was determined by the measurement of blood glucose weekly. As controls, age matched NOD scid mice received splenocytes (1.5×10⁷) from PBS treated NOD mice. Diabetes development in the NOD-scid mice was determined by detecting a blood glucose≧11.1 mmol/1.

### In Vivo Proliferative Responses of BDC2.5NOD T Cells

BDC2.5 CFSE (5,6-carboxyfluorescein diacetate succinimidylester)-spleen cell adoptive transfer experiments were performed using PDX-treated, GST-treated or PBS-treated age matched mice as recipient mice. Spleen cells (5×10⁷/ml) from BDC2.5 mice expressing transgenic TCR with specificity for an islet antigen were incubated with 5 mM CFSE at 37 degree for 30 min, washed in PBS and resuspended in complete medium. 1×10⁷ CFSE-labelled T cells were injected intravenously into the PDX-treated, GST-treated or PBS-treated NOD mice. Five days later, inguinal lymph node cells and pancreatic lymph nodes were harvested and analysed by cell flow cytometry.

### Histology and Insulin Staining

Female NOD mice immunized to either Mut Pdx1 or GST underwent pancreatic histological studies. NOD mice at the age of seven weeks were injected subcutaneously with Mut Pdx1 protein or GST daily for 4 weeks, and subsequently twice a week for 4 weeks. NOD mice that had been immunised with Mut Pdx1 protein or GST protein for 8 weeks were killed and the pancreas was removed. Eight weeks after the first treatment, five mice of each group were killed, and their pancreases were removed, fixed with 10% buffered formalin, and embedded in paraffin. Samples were cut into paraffin sections (5 μm) and placed on slides.

Tissue sections of the islet stained with hematoxylin and eosin or with anti-insulin antibodies. The slides were coded and an insulitis score was determined. Insulitis severity was histologically graded for 10-20 islets per mouse. The slides were coded and an insulitis score was determined by three independent examiners. Insulitis grade was determined as follows: 0, normal islet; 1, mononuclear infiltration, largely in the periphery, in less than 25% of the islet; 2, 25%-50% of the islet showing mononuclear infiltration; 3, over 50% of the islet showing mononuclear infiltration; 4, small, retracted islet with few mononuclear cells. Insulin and glucagon staining assay were also performed.

pCMV-RenLuc-Pdx1 Plasmid Construction

Pdx1 gene coding sequences (mouse or human) were cloned into the pREN2(12) expression vector downstream of the renilla luciferase coding sequence (lacking stop codon) using HindIII/BamHI restriction sites for expression in mammalian cells. The renilla luciferase-Pdx1 fusion gene was then cloned into pET28b for expression in E. coli. Stop codons were introduced at the 3′ end of the luciferase gene in each plasmid to make luciferase only expression controls. Renilla luciferase-GAD65 and renilla luciferase—IA2 fusion plasmids have been described (Burbelo et al., BMC Biotechnol. 2005 Aug. 18; 5:2; Burbelo et al. (2008) Diabetes Care 31, 1824-1826). All plasmids were purified using a commercially available plasmid purification kit (Plasmid Maxi Kit; Qiagen).

### RenLuc-hPdx1 Fusion Protein Lysate

Mammalian fusion protein lysates were prepared by transfecting human embryonic kidney (293) cells with each plasmid using Lipofectamine 2000 Reagent (Invitrogen) according to manufacturer's protocol in 10 cm² culture dishes. Following transfection (48 hrs), lysates were harvested using 1 ml Passive Lysis Buffer (Promega) per dish. Supernatant (lysate) was collected following centrifugation. 293 cells were cultured at 37° C. in DMEM containing 10% FBS and 1% Pen/Step.

E. coli fusion protein lysates were prepared by growing 20 ml cultures overnight and then inoculating 500 ml cultures the following day. Following 5 hr incubation at 37° C., protein expression was induced with IPTG and cultures were transferred to 30° C. for 48 hrs. Cells were then centrifuged and lysates were prepared from each culture by incubating overnight with 10 ml Passive Lysis Buffer (Promega) at 4° C. with a stir bar. DNAse was added to reduce viscosity. Supernatant (lysate) was collected following centrifugation.

### Human Serum Samples

Sera consisted of samples that were determined as positive or negative for ICA, GADA, or IA-2A, by clinical assay. Sera were also used from patients with recent onset (RO) T1D (within 6 months of onset), long standing (LS) T1D, rheumatoid arthritis, systemic lupus erythematosus, or various forms of cancer. Normal human sera obtained from 10 healthy donors were used as controls.

### Luciferase Immunoprecipitation System (LIPS) Assay

LIPS assays were performed similarly to the previously published protocols (Burbelo et al., BMC Biotechnol. 2005 Aug. 18; 5:22.; Burbelo et al., Biochem Biophys Res Commun. 2007 Jan. 26; 352(4):889-95). All sera were measured blindly. Fusion protein lysate (≧20×10⁶ RLUs in Berthold Lumat LB9507) was incubated with 10 ul human sera for PAA (or 1 ul sera for GADA and IA-2A) in 96-well round-bottom plates at a total volume of 100 ul in PBS overnight with rotation. Samples were then transferred to 96-well filter plates containing 10 ul Immobilized Protein A/G Plus (Pierce) and incubated at 4° C. for 2 hrs with rotation. All samples were washed 8 times with Buffer A as previously described (10;11) 20 ul PBS was added to each well before reading in a LUMIstar Omega plate reader (BMG Labtech). Competition assays used Pdx1 purified protein (15) or BSA at indicated concentrations and were incubated with lucifersase-Pdx1 fusion lysate overnight.

### Statistical Analysis

Statistical analysis was carried out using the two-sample Student's t-test assuming unequal variances. A P-value<0.05 was considered significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. An isolated Pdx-1 peptide comprising an amino acid sequence having about 85% identity to a Pdx-1 C terminus comprising about 50-200 or 200-283/284 Pdx-1 amino acids from the C terminus of Pdx-1 that binds an autoantibody and/or stimulates T cell proliferation.

2.-7. (canceled)

8. An isolated nucleic acid molecule encoding the Pdx-1 peptide of claim 1.

9-11. (canceled)

12. An expression vector comprising a nucleic acid sequence encoding the Pdx-1 peptide of claim 1 positioned for expression.

13.-16. (canceled)

17. A host cell comprising the expression vector of claim 12.

18.-19. (canceled)

20. A pharmaceutical composition comprising an effective amount of a Pdx-1 peptide of claim 1 or nucleic acid molecule of claim 8 and a pharmaceutically acceptable excipient.

21. An immunogenic composition or vaccine comprising a Pdx-1 polypeptide or fragment thereof in an amount sufficient to modulate an immune response and a pharmaceutically acceptable excipient.

22. (canceled)

23. (canceled)

24. A pharmaceutical composition comprising an effective amount of a Pdx-1 peptide having immunomodulatory activity in a pharmaceutically acceptable carrier.

25.-27. (canceled)

28. A method for identifying a subject as having or having a propensity to develop diabetes, the method comprising detecting a Pdx-1 autoantibody in a biological sample of the subject.

29.-31. (canceled)

32. A method for monitoring pre-diabetes or diabetes in a subject, the method comprising detecting a Pdx-1 specific antibody in a biological sample of the subject.

33. The method of claim 32, wherein a decrease in Pdx-1 specific antibody level relative to a reference indicates an improvement in pre-diabetes or diabetes in said subject.

34.-42. (canceled)

43. A method of preventing or treating pre-diabetes or diabetes in a subject, the method comprising administering to said subject an effective amount of a Pdx-1 peptide of claim 1 or a polynucleotide encoding said peptide.

44. The method of claim 43, wherein the Pdx-1 peptide is delivered for a time and in an amount sufficient to modulate an immune response in a subject identified as having Pdx-1 autoantibodies.

45. The method of claim 43, wherein the subject is identified as having an increase in Pdx-1, GAD65, IA-2, and/or insulin autoantibodies.

46. A method of inducing immunological tolerance in a subject, the method comprising administering to a subject identified as having an increase in Pdx-1, GAD65, IA-2, and/or insulin autoantibodies an effective amount of a Pdx-1 polypeptide or peptide.

47. (canceled)

48. A method of treating or preventing pre-diabetes or diabetes, said method comprising administering to a subject in need of such treatment an effective amount of a pharmaceutical composition comprising an expression vector comprising a nucleic acid molecule encoding a Pdx-1 peptide.

49.-53. (canceled)

54. A method of inducing immunological tolerance in a subject, the method comprising
(a) identifying the subject as having GAD65, IA-2, and/or insulin autoantibodies autoantibodies; and
(b) administering to the subject an effective amount of a Pdx-1 polypeptide or a nucleic acid molecule encoding a Pdx-1 polypeptide or peptide.

55.-57. (canceled)

58. A kit for use in identifying a subject as having a propensity to develop diabetes, the kit comprising a peptide of any of claim 1, and written instructions for the use of the kit in diagnosing diabetes.

59.-60. (canceled)

61. A method for diagnosing pancreatic cancer, the method comprising detecting a Pdx-1 autoantibody in a biological sample of the subject, thereby diagnosing the subject as having pancreatic cancer.

62. (canceled)

63. A method for diagnosing autoimmune disease in a subject, the method comprising detecting a Pdx-1 autoantibody in a biological sample of the subject, thereby diagnosing the subject as having an autoimmune disease.

64. The method of claim 63, wherein the autoimmune disease is rheumatoid arthritis or systemic lupus erythematosus.
